(19) United States
(12) Patent Application Publication
(10) Pub. No.: US 2025/0383362 A1
(43) Pub. Date: Dec. 18, 2025

(54) MASS SPECTROMETRIC ANALYSIS OF BIOMARKERS

(71) Applicant: IDEXX Laboratories, Inc., Westbrook, ME (US)

(72) Inventors: E.A. Prabodha Ekanayaka, Scarborough, ME (US); Jordan Haddock, Portland, ME (US); Murthy V.S.N. Yerramilli, Scarborough, ME (US)

(73) Assignee: IDEXX Laboratories, Inc., Westbrook, ME (US)

(21) Appl. No.: 19/314,548

(22) Filed: Aug. 29, 2025

Related U.S. Application Data
(62) Division of application No. 17/673,876, filed on Feb. 17, 2022.

(51) Int. Cl. G01N 33/68; G01N 30/02; G01N 30/14; G01N 30/72; G01N 30/88; G01N 33/62; G01N 33/70

(52) U.S. Cl. CPC G01N 33/6893; G01N 30/14; G01N 30/7233; G01N 30/88; G01N 33/62; G01N 33/6848; G01N 33/70; G01N 2030/027; G01N 2030/8813; G01N 2800/085; G01N 2800/347

(57) ABSTRACT

The present invention relates to methods for detecting and/or measuring alanine transaminase (ALT) activity, aspartate transaminase (AST) activity, alkaline phosphatase (ALP) activity, glucose levels, creatinine levels, urea levels, asymmetric dimethylarginine (ADMA) levels, and/or symmetrical dimethylarginine (SDMA) levels in a sample. The method allows all of the activities and analytes, or any combination of activities and analytes, to be measured in a single assay.

### CROSS-REFERENCE TO RELATED APPLICATIONS

This application is a continuation application of U.S. patent application Ser. No. 17/673,876, filed on Feb. 17, 2022, which claims the benefit of U.S. Provisional Application Ser. No. 63/151,077, filed on Feb. 19, 2021.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

The invention relates to methods for detecting and/or measuring alanine transaminase (ALT) activity, aspartate transaminase (AST) activity, alkaline phosphatase (ALP) activity, glucose levels, creatinine levels, urea levels, asymmetric dimethylarginine (ADMA) levels, and/or symmetrical dimethylarginine (SDMA) levels in a sample using mass spectrometry.

### DESCRIPTION OF RELATED ART

Serum levels of the enzymes alanine transaminase (ALT), aspartate transaminase (AST), and alkaline phosphatase (ALP) are important biomarkers for assessing organ function, such as liver and kidney function.

ALT is a pyridoxal phosphate-dependent transaminase that catalyzes the transfer of an amino group from L-alanine to α-ketoglutarate, the products of this reversible transamination reaction being pyruvate and L-glutamate.

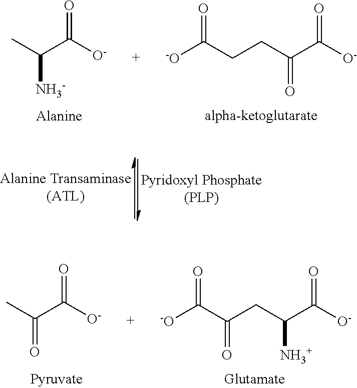

ALT is found in various body tissues, but is most common in the liver. Normal ALT serum levels typically range from 29 to 33 units/L for human males and 19 to 25 units/L for human females. Normal ALT values for canines typically ranges from 17 to 95 units/L, for felines typically ranges from 28 to 109 units/L, and for mice typically ranges from 19 to 176 units/L

AST is a pyridoxal phosphate-dependent transaminase that catalyzes the reversible transfer of an α-amino group from aspartate to α-ketoglutarate. The products of this reversible transamination reaction being oxaloacetate and glutamate.

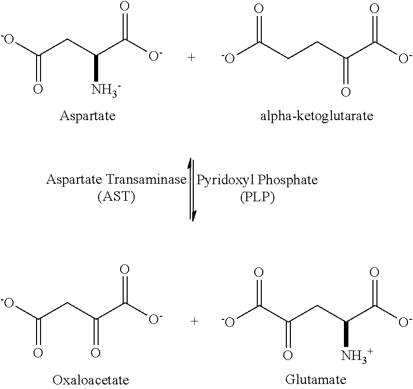

AST is found in the liver, heart, skeletal muscle, kidneys, brain, and red blood cells. Normal AST serum levels typically range from 10 to 40 units/L for human males and 9 to 32 units/L for human females. Normal AST levels for canines typically range from 18 to 56 units/L, for felines typically range from 17 to 48 units/L, and for mice typically range from 35 to 268 units/L.

Serum ALT level, serum AST level, and their ratio (AST/ALT ratio) are routinely measured clinically as biomarkers for liver health. Elevated serum levels of ALT can indicate the existence of a medical problem such as viral hepatitis, diabetes, congestive heart failure, liver damage, bile duct problems, infectious mononucleosis, or myopathy.

If elevated ALT levels are found in the blood, the possible underlying causes can be further narrowed down by measuring other enzymes. Although elevated ALT and AST levels can be associated with liver problems, elevated ALT levels are more likely related to liver injury than abnormal AST levels. In fact, if AST levels are abnormal and ALT levels are normal, the problem is much more likely due to a heart condition or muscle problem, rather than a liver problem. Myopathy-related elevations in ALT should be suspected when AST levels are greater than ALT levels; the possibility of muscle disease causing elevations in liver tests can be further explored by measuring muscle enzymes, including creatine kinase. Elevated ALT levels due to hepatocyte damage can be distinguished from bile duct problems by measuring alkaline phosphatase levels.

Alkaline phosphatase (ALP) is an enzyme that catalyzes the dephosphorylation of compounds. ALP is homodimeric enzyme, requiring three metal ions (two Zn and one Mg) for activity. The liver is the main sources of ALP, but ALP is also made in bones, intestines, pancreas, and kidneys. In pregnant women, ALP is made in the placenta.

Serum ALP levels are measured clinically as biomarkers to evaluate liver function and gall bladder function. Higher-than-normal levels of ALP in blood can indicate a problem with your liver or gallbladder, such as hepatitis, cirrhosis, liver cancer, gallstones, or a blocked bile duct. Higher-than-normal levels of ALP in blood can also indicate bone problems, such as rickets, osteomalacia, and Paget's disease. In humans, normal ALP serum levels typically range from from 20 to 140 units/L. Normal ALP levels for canines typically range from 7 to 115 units/L. Normal ALP levels for felines typically range from 11 to 49 units/L. Normal ALP levels for mice typically range from 26 to 171 units/L.

Other important biomarkers for assessing organ damage, such as kidney damage, include blood levels of creatinine, urea, glucose, ADMA, and SDMA.

Creatinine is a waste product produced by muscles from the breakdown of creatine. Creatinine is removed from the body by the kidneys, which filters it from the blood and releases it into the urine to be excreted. Serum creatinine levels are a measure of kidney function. High creatinine levels is indicative of kidney failure or kidney disease. In humans, normal creatinine levels typically range from 0.9 to 1.3 mg/dL in adult men and from 0.6 to 1.1 mg/dL in adult women. In canines, creatinine levels typically range from 0.6 to 1.4 mg/dL and in felines creatinine levels typically range from 0.8 to 2.1 mg/dL. In mice, creatinine levels typically range from 0.1 to 0.4 mg/dL.

Urea is a metabolic by-product that can also build up in the blood if kidney function is impaired. Serum urea levels are typically reported as the nitrogen component of urea, i.e., as blood urea nitrogen or BUN. BUN levels are roughly one-half (0.446) of blood urea levels. In humans, normal BUN levels typically range from 8 to 24 mg/dL for adult men, 6 to 21 mg/dL for adult women, and 7 to 20 mg/dL for children 1 to 17 years old. For canines BUN levels typically range from 9 to 31 mg/dL and for felines BUN levels typically range from 17 to 35 mg/dL range. In mice, BUN levels typically range from 17 to 39 mg/dL.

The ratio of BUN to creatinine is a measure of kidney function. The normal BUN: creatinine ratio ranges from 10:1 to 20:1. A higher ratio can be indicative of insufficient blood flow to your kidneys, such as from congestive heart failure, dehydration, or gastrointestinal bleeding. A lower ratio can be indicative of liver disease or malnutrition.

SDMA is a metabolite of L-arginine. Elevated blood SDMA levels are another indicator of kidney function. An elevated SDMA level is a reflection of impaired glomerular filtration rate (GFR). SDMA levels are used to evaluate kidney function in cats and dogs. An SDMA level greater than 14 μg/dL in cats and adult dogs or greater than 16 μg/dL in puppies can be indicative of kidney disease or kidney failure. SDMA levels in humans is typically 14 μg/dL or less.

ADMA is also a metabolite of L-arginine. ADMA is an inhibitor of NO synthesis. Elevated levels of ADMA impair endothelial function and, thus, are a risk factor atherosclerosis. Increased ADMA levels are associated with hypercholesterolemia, atherosclerosis, hypertension, chronic heart failure, and chronic renal failure. Elevated blood ADMA levels can also be indicative of pre-diabetes/diabetes. ADMA levels for cats typically range from 23 to 152 μg/dL. ADMA levels for canines typically range from 42 to 180 μg/dL.

Blood glucose levels are routinely measured to evaluate diabetes. Diabetes is an inability of the pancreas to produce insulin (Type 1 diabetes), an inability of cells to use insulin (Type 2 diabetes), or both. In humans, a fasting blood sugar level less than 100 mg/dl (5.6 mmol/L) is normal; a fasting blood sugar level from 100 to 125 mg/dL (5.6 to 6.9 mmol/L) is considered to indicate prediabetes; and a fasting blood sugar level of 126 mg/dL (7 mmol/L) or higher on two separate tests is considered indicative of diabetes. For canines blood glucose levels typically range from 68 to 104 mg/dL. For felines blood glucose levels typically range from 71 to 182 mg/dL. In mice, blood glucose levels typically range from 68 to 277 mg/dL

AST activity, ALT activity, ALP activity, and blood levels of creatinine, urea, SDMA, ADMA, and glucose are important indicators of an animal's health. Also, the dosing of drugs needs to be adjusted for patients who have renal and/or hepatic insufficiency. Thus, methods for rapidly and accurately measuring these biological markers for renal and/or hepatic function is important in clinical practice.

There remains a need in the art for methods to rapidly and accurately measuring these biological markers for renal and/or hepatic function. The present invention is directed to methods for identifying and/or quantifying these biomarkers.

These and other features and advantages of the present invention will become apparent from the remainder of the disclosure, in particular the following detailed description of the preferred embodiments, all of which illustrate by way of example the principles of the invention.

Citation of any reference in this application is not to be construed that such reference is prior art to the present application.

### SUMMARY OF THE INVENTION

The invention is directed to methods for detecting and/or measuring alanine transaminase (ALT) activity, aspartate transaminase (AST) activity, alkaline phosphatase (ALP) activity, glucose levels, creatinine levels, urea levels, asymmetric dimethylarginine (ADMA) levels, and/or symmetrical dimethylarginine (SDMA) levels in a sample using mass spectrometry.

The method for assaying alanine transaminase activity in a sample comprises:
(i) providing a sample suspected of containing alanine transaminase;
(ii) contacting the sample with a substrate for alanine transaminase, wherein the substrate comprises an isotopic label, to provide an assay mixture;
(iii) passing the assay mixture through an HPLC column to provide an eluant containing components of the assay mixture;
(iv) analyzing at least a portion of the eluant with a mass spectrometer, wherein the mass spectrometer is operated in the negative ion mode, to generate and detect an ion formed from a metabolite resulting from the action of the alanine transaminase on the substrate, wherein the metabolite comprises the isotopic label.

The method for assaying aspartate transaminase activity in a sample comprises:
(i) providing a sample suspected of containing aspartate transaminase;
(ii) contacting the sample with a substrate for aspartate transaminase, wherein the substrate comprises an isotopic label, to provide an assay mixture;
(iii) passing the assay mixture through an HPLC column to provide an eluant containing components of the assay mixture;
(iv) analyzing at least a portion of the eluant with a mass spectrometer, wherein the mass spectrometer is operated in the negative ion mode, to generate and detect an ion formed from a metabolite resulting from the action of the aspartate transaminase on the substrate, wherein the metabolite comprises the isotopic label.

The method for assaying alkaline phosphatase activity in a sample comprises:
(i) providing a sample suspected of containing alkaline phosphatase;
(ii) contacting the sample with a substrate for alkaline phosphatase to provide an assay mixture;
(iii) passing the assay mixture through an HPLC column to provide an eluant containing components of the assay mixture;
(iii) analyzing at least a portion of the eluant with a mass spectrometer, wherein the mass spectrometer is operated in the negative ion mode, to detect the presence of a metabolite resulting from the action of the alkaline phosphatase on the substrate.

The method for assaying for glucose levels comprises:
(i) providing a sample suspected of containing glucose;
(ii) adding isotopically labelled glucose to the sample, wherein the isotopically labelled glucose contains an isotopic label, to provide an assay mixture;
(iii) using mass spectrometry operated in the negative ion mode to generate and detect (a) an ion formed from the glucose and (b) an ion formed from the isotopically labelled glucose, wherein the ion formed from the isotopically labelled glucose comprises the isotopic label.

The method for assaying for urea levels comprises:
(i) providing a sample suspected of containing urea;
(ii) adding isotopically labelled urea to the sample, wherein the isotopically labelled urea contains an isotopic label, to provide an assay mixture;
(iii) using mass spectrometry operated in the positive ion mode to generate and detect (a) an ion formed from the urea and (b) an ion formed from the isotopically labelled urea, wherein the ion formed from the isotopically labelled urea comprises the isotopic label.

The method of assaying for creatinine levels comprises:
(i) providing a sample suspected of containing creatinine;
(ii) adding isotopically labelled creatinine to the sample, wherein the isotopically labelled creatinine contains an isotopic label, to provide an assay mixture;
(iii) using mass spectrometry operated in the positive ion mode to generate and detect (a) an ion formed from the creatinine and (b) an ion formed from the isotopically labelled creatinine, wherein the ion formed from the isotopically labelled creatinine comprises the isotopic label.

The method of assaying for ADMA levels comprises:
(i) providing a sample suspected of containing ADMA;
(ii) adding isotopically labelled ADMA to the sample, wherein the isotopically labelled ADMA contains an isotopic label, to provide an assay mixture;
(iii) using mass spectrometry operated in the positive ion mode to generate and detect (a) an ion formed from the ADMA and (b) an ion formed from the isotopically labelled ADMA, wherein the ion formed from the isotopically labelled ADMA comprises the isotopic label.

The method of assaying for SDMA levels comprises:
(i) providing a sample suspected of containing SDMA;
(ii) adding isotopically labelled SDMA to the sample, wherein the isotopically labeled SDMA contains an isotopic label, to provide an assay mixture;
(iii) using mass spectrometry operated in the positive ion mode to generate and detect (a) an ion formed from the SDMA and (b) an ion formed from the isotopically labelled SDMA, wherein the ion formed from the isotopically labelled SDMA comprises the isotopic label.

### BRIEF DESCRIPTION OF THE DRAWINGS

Not applicable.

### DETAILED DESCRIPTION OF THE INVENTION

### Definitions

The term “substrate,” as used herein, has its ordinary meaning in the biochemical arts, i.e., a molecule upon which an enzyme acts.

The term “metabolite,” as used herein, has its ordinary meaning in the biochemical arts, i.e., a molecule that is the product resulting from action of the enzyme on a substrate.

For example, the enzyme aspartate transaminase catalyzes the conversion of aspartate to oxaloacetate. Aspartate is the “substrate” for the enzyme and oxaloacetate is the “metabolite.” The catalytic reaction also requires the presence of α-ketoglutarate and pyridoxal phosphate. α-Ketoglutarate is a co-substrate and pyridoxal phosphate is a “cofactor,” i.e., a substance (other than the substrate) whose presence is essential for the activity of an enzyme.

The term “isotope,” as used herein, has its ordinary meaning in the chemical arts, i.e., one of two or more species of atoms of an element with the same atomic number and position in the periodic table but with different atomic masses. Isotopes of an element have an identical number of protons and electrons, but a different number of neutrons, and therefore have different atomic masses.

The phrase “isotopic label,” as used herein, means an isotope that, when incorporated into a molecule, produces a mass shift in the molecule (i.e., the isotopically labeled molecule) relative to a molecule that does not include the isotope (i.e., an unlabeled molecule) when analyzed by a mass spectrometric technique. For example, if an “isotopic label” has one additional neutron, and one isotopic label is incorporated into a molecule, the resulting isotopically labeled molecule will have a molecular weight that is increased by one mass unit relative to the unlabeled molecule.

Typically, the “isotopic label” is the isotope that has a higher atomic mass than the atomic mass of the naturally occurring isotope. Typically, the “isotopic label” is a stable isotope, i.e., an isotope whose mass does not change over time. A stable isotope is not radioactive.

Illustrative “isotopic labels” include, but are not limited to, ¹³C to replace 12C, ¹⁵N to replace ¹⁴N, ²H to replace ¹H, ¹⁷O or ¹⁸O to replace ¹⁶O, and ³⁴S to replace ³²S. Examples of a molecule comprising an “isotopic label” include, but are not limited to, a molecule wherein one or more of the carbon atoms are replaced with ¹³C atoms, one or more of the nitrogen atoms are replaced with ¹⁵N, and/or one or more of the hydrogens are replaced with ²H atoms.

As used herein, the term “high performance liquid chromatography” or “HPLC” (also sometimes known as “high pressure liquid chromatography”) refers to liquid chromatography in which the components of a sample are separated by using pressure to force a liquid mobile phase containing the sample through a solid stationary phase.

The term “mass spectrometry” (or simply “MS”), as used herein, encompasses any spectrometric technique or process in which molecules are ionized and separated and/or analyzed based on their respective molecular weights. Mass spectrometry encompasses any type of ionization method, including, but not limited to, electrospray ionization (ESI), atmospheric-pressure chemical ionization (APCI) and other forms of atmospheric pressure ionization (API), and laser irradiation.

Mass spectrometers are routinely combined with separation methods, such as gas chromatography (GC) and HPLC. GC and HPLC separates the components of a mixture, and the separated components are then individually introduced into the mass spectrometer; such techniques are generally referred to as GC/MS and LC/MS, respectively.

Multiple reaction monitoring (“MRM”) is a mass spectrometric technique that involves monitoring the formation of specific fragment ions (i.e., daughter ions) from a specified parent ion under collision induced dissociation conditions in a mass spectrometer. Using a triple quadrupole mass spectrometer, the first quadrupole acts as a filter to separate specified parent ions, i.e., ions having a specified m/z-ratio, from other parent ions. The separated parent ions are then passed into the second quadrupole, which acts as a collision chamber where the energized parent ions are collided with neutral molecules resulting in the formation of fragment ions (i.e., daughter ions). The daughter ions are then passed onto the third quadrupole where the daughter ions are separated to allow only specified daughter ions, having a specified m/z ratio, to reach a detector and record a signal. The second fragmentation step makes it possible to identify and separate ions that have very similar m/z-ratios in regular mass spectrometers. The mass spectrometer can be operated in negative ion mode (negatively charged ions are detected) or positive ion mode (positively charged ions are detected).

As used herein, the phrase “internal standard” means a compound, different from the compound being analyzed for in an assay (i.e., the analyte), that is added in a known amount to a sample containing the analyte. The signal from the analyte is compared with signal from the standard to quantify the amount of analyte in the sample.

### General Description of the Assay Method

The invention relates to methods for detecting and/or measuring alanine transaminase (ALT) activity, aspartate transaminase (AST) activity, alkaline phosphatase (ALP) activity, glucose levels, creatinine levels, urea levels, asymmetric dimethylarginine (ADMA) levels, and symmetrical dimethylarginine (SDMA) levels in a sample using mass spectrometry.

In one embodiment, the method allows any combination of ALT activity, AST activity, ALP activity, glucose levels, creatinine levels, urea levels, ADMA levels, and/or SDMA levels to be simultaneously measured in a single assay.

In one embodiment, the assay involves passing an assay mixture formed from the sample through an HPLC column to provide an eluant containing the components of the assay mixture and at least a portion of the eluant is then introduced into the mass spectrometer. The mass spectrometer analyzes the eluted components using the technique of multiple reaction monitoring or MRM.

Using MRM, a signal is detected only when a specified daughter ion is detected from a specified parent ion. Before the assay is conducted, the mass spectrometer is configured to detect specific daughter ions derived from specific parent ions (i.e., a specific daughter/parent ion). This allows compounds that generate daughter ions with same m/z to be detected and measured as long as their parent ions are different and vice versa.

In the assay method, the mass spectrometer typically spends around 50 mS (can be as low as 10 mS) monitoring for each daughter/parent ion that the mass spectrometer has been configured to detect. For example, as is discussed further below, the assay method can involve detecting seven daughter/parent ions in the negative ion mode and detecting ten daughter/parent ions in the positive ion mode. The seven daughter/parent ions that are detected in the negative ion mode are itemized in Table 1. Also provided in Table 1 is the source of each daughter/parent ion.

TABLE 1
Parent ion/daughter ion pairs detected in the negative ion mode according to the method

| | Parent ion | Daughter ion | |
|---|---|---|---|
| Source | (m/z) | (m/z) | Peak designation^{a} |
| 4-nitrophenol generated from the substrate | 138 | 46 | 46/138 |
| for ALP (also referred to as the ALP | | | |
| product) | | | |
| 4-nitrophenol standard | 144 | 46 | 46/144 |
| Pyruvate generated from the substrate for | 90 | 45 | 45/90 |
| AST (also referred to as the AST product) | | | |
| Pyruvate generated from the substrate for | 88 | 43 | 43/88 |
| ALT (also referred to as the ALT product) | | | |
| Pyruvate internal standard | 89 | 44 | 44/89 |
| Glucose | 179 | 119 | 119/179 |
| Glucose internal standard | 185 | 123 | 123/185 |

^{a}see discussion below

The ten daughter/parent ions that are detected in the positive ion mode are itemized in Table 2. Also provided in Table 2 is the source of each daughter/parent ion.

TABLE 2
Parent ion/daughter ion pairs detected in the positive ion mode according to the method

| | Parent ion | Daughter ion | |
|---|---|---|---|
| Source | (m/z) | (m/z) | Peak designation^{a} |
| Creatinine | 114 | 44 | 44/114 |
| | 114 | 86 | 86/114 |
| Creatinine internal standard | 117 | 47 | 47/117 |
| | 117 | 89 | 89/117 |
| Urea | 61 | 44 | 44/61 |
| Urea internal standard | 64 | 46 | 46/64 |
| ADMA | 203 | 46 | 46/203 |
| ADMA internal standard | 210 | 46 | 46/210 |
| SDMA | 203 | 172 | 172/203 |
| SDMA internal standard | 209 | 175 | 175/209 |

^{a}see discussion below

As noted above, the mass spectrometer typically spends about 50 mS monitoring each daughter/parent ion. After the spectrometer completes one cycle of monitoring (e.g., about 350 mS total for the seven daughter/parent ions that are detected in the negative ion mode) the cycle repeats. This cycling continues for the entire chromatographic run time of the assay.

In one embodiment, the following HPLC conditions are used:
An Atlantis HILIC Column, 100 Å, 3 μm, 2.1 mm×100 mm (commercially available from Waters Corporation of Milford, MA). The column is eluted using the following gradient:
Mobile Phase A: 20 mM Ammonium formate in water, pH 3.5.
Mobile Phase B: 100% Acetonitrile.
Initial condition-95% B, 5% A; 0.1 min-95% B, 5% A; 1.3 min-30% B, 70% A; 1.4 min-95% B, 5% A; 2.5 min-95% B, 5% A; and stop the run, wherein the changes in solvent between timepoints was carried out using a linear gradient, and the flow rate was about 0.8 mL/min. (referred to hereinafter as HPLC conditions A);
   or
Initial condition-80% B, 20% A; 0.1 min-80% B, 20% A; 0.7 min-80% B, 20% A; 0.71 min-30% B, 70% A; 1.2 min-30% B, 70% A; 1.21 min-80% B, 20% A; 2.5 min-80% B, 20% A; and stop the run wherein the changes in solvent between timepoints was carried out using a linear gradient, and the flow rate was about 1.0 mL/min (referred to hereinafter as HPLC conditions B).

In each case the chromatographic run time is about 2.5 min and the above mentioned monitoring is repeated continually throughout the 2.5 min run time (e.g., about every 350 mS for the seven daughter/parent ions that are detected in the negative ion mode). Because the mass spectrometer detects ions at a much faster rate than the chromatographic elution of compounds, using the technique of MRM allows the detection of co-eluting molecules. For example, the assay method involves detecting three different forms of pyruvate (pyruvate generated from the substrate for AST, pyruvate generated from the substrate for ALT, and a pyruvate internal standard) in the negative ion mode. All three forms of pyruvate have same retention time and the chromatographic peak width for pyruvate is about 0.2 min (12 sec). Since the mass spectrometer goes through one cycle of monitoring every 350 mS, it is able to detect and measure all three forms of pyruvates as they elute, even though they are co-eluting.

Below is described in more detail the assay for detecting and/or measuring alanine transaminase (ALT) activity, aspartate transaminase (AST) activity, alkaline phosphatase (ALP) activity, glucose levels, creatinine levels, urea levels, asymmetric dimethylarginine (ADMA) levels, and symmetrical dimethylarginine (SDMA) levels in a sample.

Importantly, because the method, wherein the mass spectrometer is operated in the negative ion mode, detects a different daughter/parent ion for each of nitrophenol generated from ALP activity, pyruvate from ALT activity, pyruvate from AST activity, and glucose the method allows ALP activity, ALT activity, AST activity, and glucose levels to be determined simultaneously.

Similarly, because the method, wherein the mass spectrometer is operated in the positive ion mode, detects a different daughter/parent ion for each of creatinine, urea, ADMA, and SDMA the method allows the levels of each of these compounds to be determined simultaneously.

### Assay for Alanine Transaminase Activity

The method for assaying alanine transaminase activity in a sample comprises:
(i) providing a sample suspected of containing alanine transaminase;
(ii) contacting the sample with a substrate for alanine transaminase, wherein the substrate comprises an isotopic label, to provide an assay mixture;
(iii) passing the assay mixture through an HPLC column to provide an eluant containing components of the assay mixture;
(iv) analyzing at least a portion of the eluant with a mass spectrometer, wherein the mass spectrometer is operated in the negative ion mode to generate and detect an ion formed from a metabolite resulting from the action of the alanine transaminase on the substrate, wherein the metabolite comprises the isotopic label.

Alanine transaminase catalyzes the conversion of alanine to pyruvate (or, depending on the pH, pyruvic acid). The reaction is depicted below:

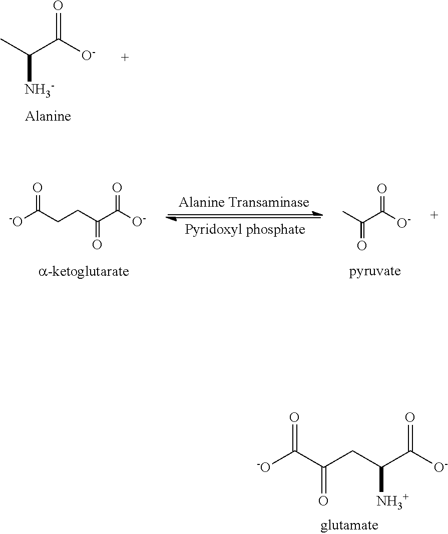

α-Ketoglutarate is a co-substrate and pyridoxal phosphate (PLP) is a cofactor for the reaction. The α-ketoglutarate is converted to glutamate. Thus, α-ketoglutarate (e.g., α-ketoglutaric acid disodium salt dihydrate, commercially available from Sigma Aldrich of St. Louis, MO) and pyridoxal phosphate (e.g., pyridoxal 5′-phosphate monohydrate, commercially available from Sigma Aldrich of St. Louis, MO) are added to the sample.

The sample can be contacted with the substrate for alanine transaminase at a pH of between about 6.0 and about 9.0, preferable between about 6.5 and about 8.5. In one embodiment, the pH is about 7.4.

Suitable buffers for maintaining the pH include, but are not limited to, a Tris buffer, an ammonium carbonate buffer, and an ammonium bicarbonate buffer. In one embodiment, the buffer is a 20-200 mM Tris buffer. In one embodiment, the buffer is 100 mM Tris with 10 mM ammonium bicarbonate.

The assay mixture (containing the alanine transaminase and the isotopically labeled substrate, along with cofactors, and buffer) are incubated for a given amount of time, typically at a constant temperature, so that the enzymatic conversion of the substrate to the metabolite can take place. After the allotted time has elapsed, the assay mixture is quenched to stop the enzymatic reaction.

In embodiment, the incubation temperature ranges from about 30° C. to about 50° C., preferably about 35° C. to 45° C., for example about 40° C., and the incubation time ranges from about 5 minutes to about 20 minutes, preferably about 7 minutes to about 15 minutes, for example about 10 minutes, before the assay mixture is quenched. In one embodiment, the assay mixture is incubated at a temperature of about 40° C. for about 10 minutes before the assay mixture is quenched.

The reaction mixture can be quenched, for example, by rapidly cooling the assay mixture, or by adding a reagent that stops the reaction, for example, by denaturing the alanine transaminase. In one embodiment, the reaction mixture is quenched by adding an organic solvent. Suitable organic solvents include acetonitrile, acetone, methanol, and mixtures thereof. In one embodiment, about 200 μL to about 600 μL of organic solvent is added to 10 μL of sample to quench the reaction mixture. In one embodiment, the reaction mixture is quenched by adding methanol. For example, about 200 μL to about 600 μL of methanol is added to 10 μL of sample to quench the reaction mixture. In one embodiment, about 300 μL of methanol is added to 10 μL of sample to quench the reaction mixture.

In one embodiment, the substrate for alanine transaminase is alanine labeled with ¹³C in the 1-position ((S)-2-ammoniopropanoate-1-¹³C), i.e.,

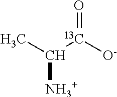

and the metabolite is pyruvate (or, depending on the pH, pyruvic acid) labeled with a ¹³C at the 1-position (2-oxopropionate-1-¹³C), i.e.,

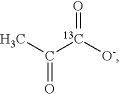

also referred to herein as the ALT product.

Alanine labeled with ¹³C in the 1-position is commercially available from Cambridge Isotope Laboratories, Inc. of Tewksbury, MA.

Pyruvate labeled with a ¹³C at the 1-position has a molecular weight of 88. When analyzed in the mass spectrometer, pyruvate labeled with a ¹³C at the 1-position forms a parent ion that has an m/z ratio of 88.

When alanine labeled with ¹³C in the 1-position is used as the substrate, the metabolite is detected by:
(i) subjecting at least a portion of the eluant to ionization in a mass spectrometer in the negative ion mode to provide a plurality of parent ions;
(ii) separating the parent ions having an m/z ratio of 88 from the plurality of parent ions;
(iii) fragmenting the parent ions having an m/z ratio of 88 to provide a plurality of daughter ions;
(iv) separating daughter ions that have an m/z ratio of 43 from the plurality of daughter ions; and
(v) detecting the intensity of the daughter ions that have an m/z ratio of 43.

A peak having an m/z ratio of 43 derived from a parent ion having an m/z ratio of 88 is indicative of alanine transaminase activity. We refer to the peak having an m/z ratio of 43 derived from a parent ion having an m/z ratio of 88 as the 43/88 peak. Hereinafter we refer to a peak having an m/z ratio of “x” that is derived from a parent ion having an m/z ratio of “y” as the “x/y peak.” The intensity of the 43/88 peak can be used to determine the amount of pyruvate formed by the alanine transaminase catalyzed conversion of alanine to pyruvate.

Without wishing to be bound by theory, it is believed that the structure of the daughter ion having an m/z ratio of 43 is:

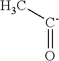

Without wishing to be bound by theory, it is believed that the fragmentation scheme to provide the daughter ion is as depicted below:

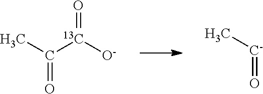

The assay can be performed using a triple quadrupole mass spectrometer wherein the first quadrupole separates the parent ions having an m/z ratio of 88 from other parent ions; the second quadrupole is a collision chamber that fragments the parent ions having an m/z ratio of 88 to provide daughter ions, and the third quadrupole separates daughter ions that have an m/z ratio of 43 from other daughter ions.

The alanine transaminase activity can be quantified by comparing the intensity of the 43/88 peak obtained from the sample with the intensity of the 43/88 peak obtained from a positive control that includes a known amount of alanine transaminase of a known activity (e.g., Alanine Aminotransferase, Porcine Heart, commercially available from LeeBio Solutions of Maryland Heights, MO) that is treated in the same way that the sample is treated. For example, if the positive control has an alanine transaminase activity of 25 units/liter and provides an intensity of 100 for the 43/88 peak and the sample provides an intensity of 50 for the 43/88 peak, the alanine transaminase activity in the sample would be 12.5 units/liter.

In one embodiment, the sample may include an internal standard. In one embodiment, the internal standard is pyruvate (or, depending on the pH, pyruvic acid) labeled with ¹³C at the 1- and 2-positions (2-oxopropionate-1, 2-¹³C), i.e.,

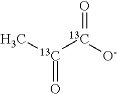

Pyruvate labeled with a ¹³C at the 1- and 2-positions is commercially available from Sigma Aldrich Inc. of St. Louis, MO.

Pyruvate labeled with a ¹³C at the 1- and 2-positions has a molecular weight of 89. When the sample is analyzed with the triple quadrupole mass spectrometer, the pyruvate labeled with a ¹³C at the 1- and 2-positions will form a parent ion that has an m/z ratio of 89, which will then undergo fragmentation in the second quadrupole to provide daughter ions in the same way that the metabolite (i.e., pyruvate labeled with a ¹³C at the 1-position) undergoes fragmentation to provide daughter ions, as depicted below:

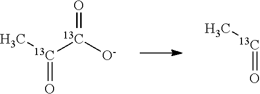

Thus, the internal standard will provide daughter ions having an m/z of 44 derived from a parent ion having an m/z ratio of 89, i.e., a 44/89 peak.

When the sample includes pyruvate labeled with a ¹³C at the 1- and 2-positions as an internal standard, the mass spectrometer is configured so that the first quadrupole separates parent ions having an m/z ratio of 88 (pyruvate formed from the substrate) and 89 (pyruvate formed from the internal standard) and the third quadrupole separates daughter ions having an m/z ratio of 43 (daughter ions derived from pyruvate formed from the substrate, i.e., CH₃C(O)⁻) and 44 (daughter ions derived from pyruvate formed from the internal standard, i.e., CH₃¹³C(O)⁻). The detector is configured to record the intensity of ions having an m/z ratio of 43 derived from a parent ion having an m/z ratio of 88 and an m/z ratio of 44 derived from a parent ion having an m/z ratio of 89, i.e., 43/88 and 44/89 peaks.

The internal standard is added to the sample in a known amount. The ratio of the intensity of the 43/88 peak to the intensity of the 44/89 peak is used to determine the amount of pyruvate formed by the alanine transaminase catalyzed conversion of alanine to pyruvate.

The alanine transaminase activity can be quantified by comparing the ratio of the intensity of the 43/88 peak to the intensity of the 44/89 peak obtained from the sample with the ratio of the intensity of the 43/88 peak to the intensity of the 44/89 peak obtained from a positive control that includes a known amount of alanine transaminase of a known activity (e.g., alanine Aminotransferase, Porcine Heart, commercially available from LeeBio Solutions of Maryland Heights, MO) that is treated in the same way as the sample is treated.

For example, if the positive control has an alanine transaminase activity of 25 units/liter and provides a value of 100 for the ratio of the intensity of the 43/88 peak to the intensity of the 44/89 peak and the sample provides a value of 50 for the ratio of the intensity of the 43/88 peak to the intensity of the 44/89 peak, the alanine transaminase activity in the sample would be 12.5 units/liter.

Thus, in one embodiment, the method for assaying alanine transaminase activity in a sample comprises:
(i) providing a sample suspected of containing alanine transaminase;
(ii) contacting the sample with

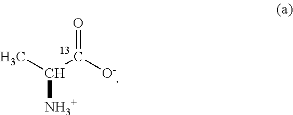

(b) α-ketoglutarate, and
(c) pyridoxal phosphate,
   to provide an assay mixture;
(iii) allowing the assay mixture to incubate at a temperature for a period of time;
(iv) quenching the assay mixture;
(v) passing the quenched assay mixture through an HPLC column to provide an eluant containing components of the assay mixture;
(vi) subjecting at least a portion of the eluant to ionization in a mass spectrometer in the negative ion mode to provide a plurality of parent ions;
(vii) separating parent ions having an m/z ratio of 88 from the plurality of parent ions;
(viii) fragmenting the parent ions having an m/z ratio of 88 to provide a plurality of daughter ions;
(ix) separating daughter ions that have an m/z ratio of 43 from the plurality of daughter ions; and
(x) detecting the intensity daughter ions that have an m/z ratio of 43.

In one embodiment, the assay further comprises adding

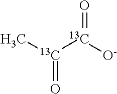

to the sample as an internal standard. When the assay comprises adding pyruvate labeled with a ¹³C at the 1- and 2-positions as an internal standard, the assay involves separating the parent ions having an m/z ratio of 88 and 89 from the plurality of parent ions and separating daughter ions having an m/z ratio of 43 and 44 from the plurality of daughter ions.

In one embodiment, the alanine transaminase activity can be quantified by creating a standard curve prepared by adding a fixed concentration of pyruvate labeled with ¹³C at the 1-and 2-positions (i.e., pyruvate internal standard) to a series of solutions containing pyruvate labeled with a ¹³C at the 1-position (i.e., ALT product) of different concentrations, determining the intensity of the 43/88 peak relative to the intensity of the 44/89 for each solution, and plotting the intensity of the 43/88 peak relative to the intensity of the 44/89 vs. concentration of the ALT product. The sample is prepared so that it includes the same concentration of internal standard. The intensity of the 43/88 peak relative to the intensity of the 44/89 from the sample is determined and the concentration of the ALT product in the sample is obtained from the standard curve. The enzyme activity, typically reported in “activity units per liter” (U/L, where U=μmol product/min), is determined according to the following equation:

$$\frac{( \frac{( μ g / mL \times 1000 )}{89 g / mol} )}{t} = U / L$$

wherein concentration (obtained from the standard curve) is in μg/mL and t is the incubation time in minutes.

In another embodiment, the substrate for alanine transaminase is alanine labeled with a ¹³C in the 1 position as well as ¹⁵N, i.e.,

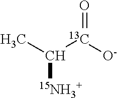

which forms pyruvate (or, depending on the pH, pyruvic acid) labeled with a ¹³C at the 1-position, i.e.,

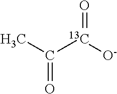

as a metabolite, i.e., the ALT product. Because alanine labeled with ¹³C in the 1 position as well as ¹⁵N forms the same metabolite as when alanine labeled with ¹³C in the 1-position is used as the substrate, the alanine transaminase activity can be determined in the same way as described above when using alanine labeled with ¹³C in the 1-position, i.e., measuring the intensity of the 43/88 peak. Alanine labeled with ¹³C in the 1-position is commercially available from Cambridge Isotope Laboratories, Inc. of Tewksbury, MA. Alanine labeled with ¹³C in the 1-position and ¹⁵N is also commercially available from Cambridge Isotope Laboratories, Inc. of Tewksbury, MA.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the quenched assay mixture is passed through an HPLC column using HPLC conditions A.

### Assay for Aspartate Transaminase Activity

The method for assaying aspartate transaminase activity in a sample comprises:
(i) providing a sample suspected of containing aspartate transaminase;
(ii) contacting the sample with a substrate for aspartate transaminase, wherein the substrate comprises an isotopic label, to provide an assay mixture;
(iii) passing the assay mixture through an HPLC column to provide an eluant containing components of the assay mixture;
(iv) analyzing at least a portion of the eluant with a mass spectrometer, wherein the mass spectrometer is operated in the negative ion mode, to generate and detect an ion formed from a metabolite resulting from the action of the aspartate transaminase on the substrate, wherein the metabolite comprises the isotopic label.

Aspartate transaminase catalyzes the conversion of aspartate to oxaloacetate (or, depending on the pH, oxaloacetic acid). The reaction is depicted below:

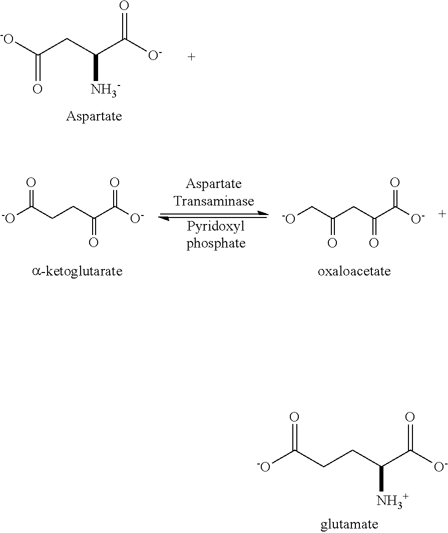

α-Ketoglutarate is a co-substrate and pyridoxal phosphate (PLP) is a cofactor for the reaction. The α-ketoglutarate is converted to glutamate. Thus, α-ketoglutarate and pyridoxal phosphate are added to the sample.

The oxaloacetate formed from the action of aspartate transaminase spontaneously decarboxylates to provide pyruvate, as depicted below:

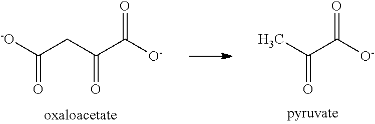

In one embodiment, oxaloacetate decarboxylase (e.g., oxaloacetate decarboxylase from Pseudomonas sp., commercially available from Sigma Aldrich of St. Louis, MO) can be added to the sample to assure that decarboxylation of oxaloacetate to provide pyruvic acid is complete.

In one embodiment, the sample is buffered to a pH ranging from about 6.0 and about 9.0, preferably between about 7.0 and about 8.0. In one embodiment, the pH is about 7.4. Suitable buffers for maintaining the pH include, but are not limited to, those described above.

The assay mixture (containing the aspartate transaminase and the isotopically labeled substrate, along with the cofactors, oxaloacetate decarboxylase, and buffer) are incubated for a given amount of time, typically at a constant temperature, so that the enzymatic conversion of the substrate to the metabolite can take place. After the allotted time has elapsed, the assay mixture is quenched to stop the enzymatic reaction.

In embodiment, the incubation temperature ranges from about 30° C. to about 50° C., preferably about 35° C. to 45° C., for example about 40° C., and the incubation time ranges from about 5 minutes to about 20 minutes, preferably about 7 minutes to about 15 minutes, for example about 10 minutes, before the assay mixture is quenched. In one embodiment, the assay mixture is incubated at a temperature of about 40° C. for about 10 minutes before the assay mixture is quenched.

The reaction mixture can be quenched, for example, by rapidly cooling the assay mixture, or by adding a reagent that stops the reaction, for example, by denaturing the alanine transaminase. In one embodiment, the reaction mixture is quenched by adding an organic solvent. Suitable organic solvents include acetonitrile, acetone, methanol, and mixtures thereof. In one embodiment, about 200 μL to about 600 μL of organic solvent is added to 10 μL of sample to quench the reaction mixture. In one embodiment, the reaction mixture is quenched by adding methanol. For example, about 200 μL to about 600 μL of methanol is added to 10 μL of sample to quench the reaction mixture. In one embodiment, about 300 μL of methanol is added to 10 μL of sample to quench the reaction mixture.

In one embodiment, the substrate for aspartate transaminase is aspartate labeled with ¹³C in the 1-, 2-, 3-, and 4-position and with ¹⁵N at the nitrogen, i.e.,

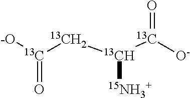

and the metabolite is oxaloacetate labeled with a ¹³C at the 1-, 2-, 3-, and 4-positions, i.e.,

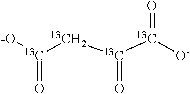

Aspartate labeled with ¹³C in the 1-, 2-, 3-, and 4-position and with ¹⁵N at the nitrogen is commercially available from Cambridge Isotope Laboratories, Inc. of Tewksbury, MA).

When the oxaloacetate labeled with a ¹³C at the 1-, 2-, 3-, and 4-positions undergoes decarboxylation it provides pyruvate (or, depending on the pH pyruvic acid) labeled with ¹³C at the 1-, 2-, and 3-positions, i.e.,

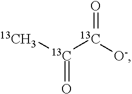

as a metabolite, also referred to herein as the AST product.

Pyruvate (labeled with ¹³C at the 1-, 2-, and 3-positions has a molecular weight of 90. When analyzed in the mass spectrometer, pyruvate labelled with ¹³C at the 1-, 2-, and 3-positions forms a parent ion that has an m/z ratio of 90.

When aspartate labeled with ¹³C in the 1-, 2-, 3-, and 4-position and with ¹⁵N at the nitrogen is used as the substrate, the metabolite is detected by:
(i) subjecting at least a portion of the eluant to ionization in a mass spectrometer in the negative ion mode to provide a plurality of parent ions;
(ii) separating the parent ions having an m/z ratio of 90 from the plurality of parent ions;
(iii) fragmenting the parent ions having an m/z ratio of 90 to provide a plurality of daughter ions;
(iv) separating daughter ions that have an m/z ratio of 45 from the plurality of daughter ions; and
(v) detecting the intensity of the daughter ions that have an m/z ratio of 45.

A mass spectrum peak at an m/z of ratio of 45 derived from parent ions having an m/z ratio of 90, i.e., a 45/90 peak, is indicative of aspartate transaminase activity. The intensity of the 45/90 peak can be used to determine the amount of pyruvate formed by the aspartate transaminase catalyzed conversion of aspartate to pyruvate.

Without wishing to be bound by theory, it is believed that the structure of the daughter ion having an m/z ratio of 45 is:

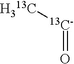

Without wishing to be bound by theory, it is believed that the fragmentation scheme to provide the daughter ion is as depicted below:

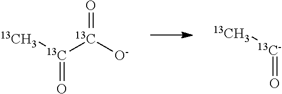

The assay can be performed using a triple quadrupole mass spectrometer wherein the first quadrupole separates the parent ions having an m/z ratio of 90 from other parent ions; the second quadrupole is a collision chamber that fragments the parent ions having an m/z ratio of 90 to provide daughter ions, and the third quadrupole separates daughter ions that have an m/z ratio of 45 from other daughter ions.

The aspartate transaminase activity can be quantified by comparing the intensity of the 45/90 peak obtained from the sample with the intensity of the 45/90 peak obtained from a positive control that includes a known amount of aspartate transaminase of known activity (e.g., Aspartate Aminotransferase, Porcine Heart, commercially available from LeeBio Solutions of Maryland Heights, MO) that is treated in the same way that the sample is treated. For example, if the positive control has an aspartate transaminase activity of 50 units/liter and provides an intensity of 100 for the 45/90 peak and the sample provides an intensity of 25 for the 45/90 peak, the aspartate transaminase activity in the sample would be 12.5 units/liter.

In one embodiment, the sample may include an internal standard. The internal standard can be pyruvate labeled with a ¹³C at the 1- and 2-positions (2-oxopropionate-1, 2-¹³C), i.e.,

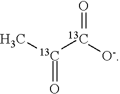

Pyruvate labeled with a ¹³C at the 1- and 2-positions has a molecular weight of 89. When the sample is analyzed with the mass spectrometer, the pyruvate labeled with ¹³C at the 1- and 2-positions will form a parent ion that has an m/z ratio of 89, which will then undergo fragmentation in the second quadrupole to provide daughter ions in the same way that the pyruvate labeled with a ¹³C at the 1-, 2-, and 3-positions (resulting from decarboxylation of the oxaloacetate labeled with a ¹³C at the 1-, 2-, 3-, and 4-positions) undergoes fragmentation to provide daughter ions. This fragmentation of the parent ion from the internal standard is depicted below:

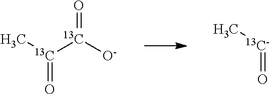

Thus, the internal standard will provide daughter ions having an m/z ratio of 44.

When the sample includes pyruvate labeled with a ¹³C at the 1- and 2-positions as an internal standard, the mass spectrometer is configured so that the first quadrupole separates parent ions having an m/z ratio of 90 (pyruvate formed from the substrate) and 89 (pyruvate from the internal standard) and the third quadrupole separates daughter ions having an m/z ratio of 45 (daughter ions derived from pyruvate formed from the substrate, i.e., ¹³CH₃¹³C(O)⁻) and 44 (daughter ions derived from pyruvate from the internal standard, i.e., CH₃¹³C(O)⁻). The detector is configured to record the intensity of ions having an m/z ratio of 45 derived from parent ions having an m/z ratio of 90 and an m/z ratio of 44 derived from parent ions having an m/z ratio of 89, i.e., the 45/90 peak and the 44/89 peak.

The internal standard is added to the sample in a known amount. The ratio of the intensity of the 45/90 peak to the intensity of the 44/89 peak is used to determine the amount of pyruvate formed by the aspartate transaminase catalyzed conversion of aspartate to pyruvate.

The aspartate transaminase activity can be quantified by comparing the ratio of the intensity of the 45/90 peak to the intensity of the 44/89 peak obtained from the sample with the ratio of the intensity of the 45/90 peak to the intensity of the 44/89 peak obtained from a positive control that includes a known amount of aspartate transaminase of a known activity (e.g., aspartate aminotransferase, porcine heart, commercially available from LeeBio Solutions of Maryland Heights, MO) that is treated in the same way as the sample is treated.

For example, if the positive control has an aspartate transaminase activity of 25 units/liter and provides a value of 100 for the ratio the intensity of the 45/90 peak relative to the intensity of the 44/89 and the sample provides a value of 50 for the ratio of the intensity of the 45/90 peak to the intensity of the 44/89 peak, the aspartate transaminase activity in the sample would be 12.5 units/liter.

Thus, in one embodiment, the method for assaying aspartate transaminase activity in a sample comprises:
(i) providing a sample suspected of containing aspartate transaminase;
(ii) contacting the sample with
   (a)

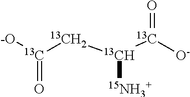

(b) α-ketoglutarate,
(c) pyridoxal phosphate, and
(d) oxaloacetate decarboxylase;
   to provide an assay mixture;
(iii) allowing the assay mixture to incubate at a temperature for a period of time;
(iv) quenching the assay mixture;
(v) passing the quenched assay mixture through an HPLC column to provide an eluant containing components of the assay mixture;
(vi) subjecting at least a portion of the eluant to ionization in a mass spectrometer in the negative ion mode to provide a plurality of parent ions;
(vii) separating parent ions having an m/z ratio of 90 from the plurality of parent ions;
(viii) fragmenting the parent ions having an m/z ratio of 90 to provide a plurality of daughter ions;
(ix) separating daughter ions that have an m/z ratio of 45 from the plurality of daughter ions; and
(x) detecting the intensity daughter ions that have an m/z ratio of 45.

In one embodiment, the assay further comprises adding

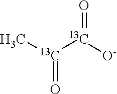

to the sample as an internal standard. When the assay comprises adding pyruvate labeled with a ¹³C at the 1- and 2-positions as an internal standard, the assay involves separating the parent ions having an m/z ratio of 90 and 89 from the plurality of parent ions and separating daughter ions having an m/z ratio of 45 and 44 from the plurality of daughter ions.

In one embodiment, the aspartate transaminase activity can be quantified by creating a standard curve prepared by adding a fixed concentration of pyruvate labeled with ¹³C at the 1- and 2-positions (i.e., pyruvate internal standard) to a series of solutions containing pyruvate labeled with ¹³C at the 1-, 2-, and 3-positions (i.e., AST product) of different concentrations, determining the intensity of the 45/90 peak relative to the intensity of the 44/89 peak for each solution, and plotting the intensity of the 45/90 peak relative to the intensity of the 44/89 vs. concentration of the AST product. The sample is prepared so that it includes the same concentration of internal standard. The intensity of the 45/90 peak relative to the intensity of the 44/89 from the sample is determined and the concentration of the AST product in the sample is obtained from the standard curve. The enzyme activity, typically reported in “activity units per liter” (U/L, where U=μmol product/min), is determined according to the following equation:

$$\frac{( \frac{( μ g / mL \times 1000 )}{91 g / mol} )}{t} = U / L$$

wherein concentration (obtained from the standard curve) is in μg/mL and t is the incubation time in minutes.

In one embodiment, the substrate for aspartate transaminase is aspartate labeled with ¹³C in the 1-, 2-, 3-, and 4-position, i.e.,

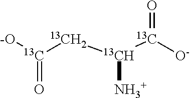

which also forms oxaloacetate labeled with a ¹³C at the 1-, 2-, 3-, and 4-positions, i.e.,

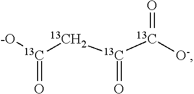

which undergoes decarboxylation to provide pyruvate (or, depending on the pH pyruvic acid) labeled with ¹³C at the 1-, 2-, and 3-positions, i.e.,

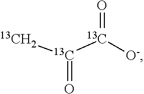

as a metabolite, i.e., the AST product. Because aspartate labeled with ¹³C in the 1-, 2-, 3-, and 4-position forms the same metabolite as when aspartate labeled with ¹³C in the 1-, 2-, 3-, and 4-position and with ¹⁵N at the nitrogen is used as the substrate, the aspartate transaminase activity can be determined in the same way as described above when using aspartate labeled with ¹³C in the 1-, 2-, 3-, and 4-position and with ¹⁵N at the nitrogen, i.e., measuring the intensity of the 45/90 peak. Aspartate labeled with ¹³C in the 1-, 2-, 3-, and 4-position is commercially available from Sigma Aldrich of St. Louis, MO and Cambridge Isotope Laboratories, Inc. of Tewksbury, MA.

In another embodiment, the substrate for aspartate transaminase is aspartate labeled with ¹³C in the 1-, 2-, and 3-position, i.e.,

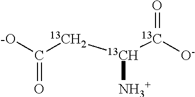

which forms oxaloacetate labeled with a ¹³C at the 1-, 2-, and 3-positions, i.e.,

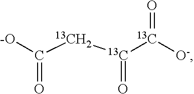

which undergoes decarboxylation to provide pyruvate (or, depending on the pH pyruvic acid) labeled with ¹³C at the 1-, 2-, and 3-positions, i.e.,

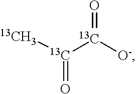

as a metabolite, i.e., the AST product. Because aspartate labeled with ¹³C in the 1-, 2-, and 3-position forms the same metabolite as when aspartate labeled with ¹³C in the 1-, 2-, 3-, and 4-position and with ¹⁵N at the nitrogen (and aspartate labeled with ¹³C in the 1-, 2-, 3-, and 4-position) is used as the substrate, the aspartate transaminase activity can be determined in the same way as described above when using aspartate labeled with ¹³C in the 1-, 2-, 3-, and 4-position and with ¹⁵N at the nitrogen, i.e., measuring the intensity of the 45/90 peak. Aspartate labeled with ¹³C in the 1-, 2-, and 3-position could readily be synthesized by a person of ordinary skill in the art.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the quenched assay mixture is passed through an HPLC column using HPLC conditions A.

### Method for Assaying Alkaline Phosphatase Activity

The method for assaying alkaline phosphatase activity in a sample comprises:
(i) providing a sample suspected of containing alkaline phosphatase;
(ii) contacting the sample with a substrate for alkaline phosphatase to provide an assay mixture;
(iii) passing the assay mixture through an HPLC column to provide an eluant containing components of the assay mixture;
(iii) analyzing at least a portion of the eluant with a mass spectrometer, wherein the mass spectrometer is operated in the negative ion mode, to detect the presence of a metabolite resulting from the action of the alkaline phosphatase on the substrate.

Alkaline phosphatase catalyzes the hydrolysis of phosphate ester substrates. An illustrative hydrolysis reaction is the hydrolysis of p-nitrophenyl phosphate to p-nitrophenol (or, depending on the pH, p-nitrophenolate), depicted below:

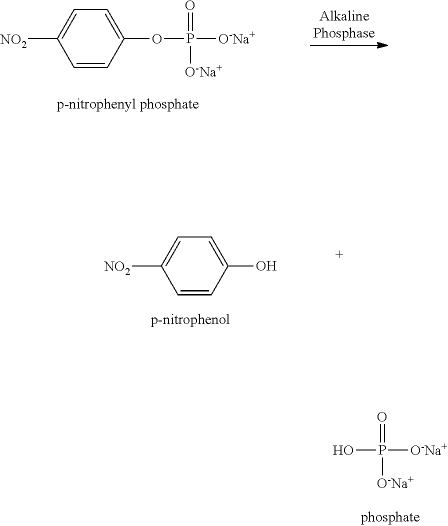

Alkaline phosphatase activity is enhanced with the addition of zinc and magnesium in the substrate solution. Thus in one embodiment, a zinc salt, a magnesium salt, and hydroxyethylethylenediaminetriacetic acid (HEDTA, commercially available from Sigma Aldrich of St. Louis, MO) are included in the alkaline phosphatase substrate solution. In one embodiment, zinc sulfate, magnesium acetate, and HEDTA are included in the alkaline phosphatase substrate solution.

The sample can be contacted with the substrate for alkaline phosphatase at a pH of between about 9.0 and about 12.0, preferable between about 9.5 and about 11.0. In one embodiment, the pH is about 10.2. Suitable buffers for maintaining the pH include, but are not limited to, 2-amino-2-methyl-1-propanol, ammonia-ammonium chloride, glycine, tricine, Tris, ethylaminoethanol, diethanolamine, and sodium carbonate-sodium bicarbonate buffers. In one embodiment, the buffer is a 2-amino-2-methyl-1-propanol buffer.

The assay mixture (containing the alkaline phosphatase and the substrate, along with buffer) are incubated for given amount of time, typically at a constant temperature, so that the enzymatic conversion of the substrate to the metabolite can take place. After the allotted time has elapsed, the assay mixture is quenched to stop the enzymatic reaction.

In embodiment, the incubation temperature ranges from about 30° C. to about 50° C., preferably about 35° C. to 45° C., for example about 40° C., and the incubation time ranges from about 5 minutes to about 20 minutes, preferably about 7 minutes to about 15 minutes, for example about 10 minutes, before the assay mixture is quenched. In one embodiment, the assay mixture is incubated at a temperature of about 40° C. for about 10 minutes before the assay mixture is quenched.

The reaction mixture can be quenched, for example, by rapidly cooling the assay mixture, or by adding a reagent that stops the reaction, for example, by denaturing the alkaline phosphatase. In one embodiment, the reaction mixture is quenched by adding an organic solvent. Suitable organic solvents include acetonitrile, acetone, methanol, and mixtures thereof. In one embodiment, about 200 μL to about 600 μL of organic solvent is added to 10 μL of sample to quench the reaction mixture. In one embodiment, the reaction mixture is quenched by adding methanol. For example, about 200 μL to about 600 μL of methanol is added to 10 μL of sample to quench the reaction mixture. In one embodiment, about 300 μL of methanol is added to 10 μL of sample to quench the reaction mixture.

In one embodiment, the substrate for alkaline phosphatase is p-nitrophenyl phosphate, e.g.,

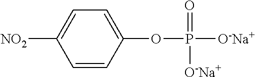

and the metabolite is p-nitrophenolate, i.e.,

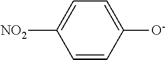

(or, depending on the pH, p-nitrophenol), also referred to as the ALP product.

p-Nitrophenyl phosphate is commercially available from Sigma Aldrich of St. Louis, MO.

p-Nitrophenolate has a molecular weight of 138. When analyzed in the mass spectrometer, the p-nitrophenolate forms a parent ion that has an m/z ratio of 138.

When p-nitrophenyl phosphate is used as the substrate, the metabolite is detected by:
(i) subjecting at least a portion of the eluant to ionization in a mass spectrometer in the negative ion mode to provide a plurality of parent ions;
(ii) separating the parent ions having an m/z ratio of 138 from the plurality of parent ions;
(iii) fragmenting the parent ions having an m/z ratio of 138 to provide a plurality of daughter ions;
(iv) separating daughter ions that have an m/z ratio of 46 from the plurality of daughter ions; and
(v) detecting the intensity of the daughter ions that have an m/z ratio of 46.

A peak at an m/z of ratio of 46 derived from a parent ion having an m/z ratio of 138, i.e., a 46/138 peak, is indicative of alkaline phosphatase activity. The intensity of the 46/138 peak can be used to determine the amount of p-nitrophenolate formed by the alkaline phosphatase catalyzed conversion of p-nitrophenyl phosphate to p-nitrophenol.

Without wishing to be bound by theory it is believed that the structure of the daughter ion having an m/z ratio of 46 is:

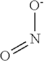

Without wishing to be bound by theory, it is believed that the fragmentation scheme to provide the daughter ion is as depicted below:

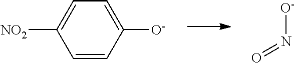

The assay can be performed using a triple quadrupole mass spectrometer wherein the first quadrupole separates the parent ions having an m/z ratio of 138 from other parent ions; the second quadrupole is a collision chamber that fragments the parent ions having an m/z ratio of 138 to provide daughter ions, and the third quadrupole separates daughter ions that have an m/z ratio of 46 from other daughter ions.

The alkaline phosphatase activity can be quantified by comparing the intensity of the 46/138 peak obtained from the sample with the intensity of the 46/138 peak obtained from a positive control that includes a known amount of alkaline phosphatase of a known activity (e.g., alkaline phosphatase from bovine intestinal mucosa, commercially available from Sigma Aldrich of St. Louis, MO) that is treated in the same way as the sample is treated. For example, if the positive control has an alkaline phosphatase activity of 25 units/liter and provides an intensity of 100 for the 46/138 peak and the sample provides an intensity of 50 for the 46/138 peak, the alkaline phosphatase activity in the sample would be 12.5 units/liter.

In one embodiment, the sample may include an internal standard. The internal standard can p-nitrophenol labeled with ¹³C at each of the carbon atoms (i.e., 4-nitrophenol ¹³C6), i.e.,

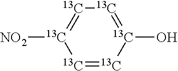

p-Nitrophenol labeled with ¹³C at each of the carbon atoms is commercially available from Sigma Aldrich of St. Louis, MO.

p-Nitrophenol labeled with ¹³C at each of the carbon atoms has a molecular weight of 145. When the sample is analyzed with the mass spectrometer, p-nitrophenol labeled will form a parent ion that has an m/z ratio of 144, i.e.,

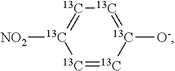

which will then undergo fragmentation in the second quadrupole to provide daughter ions in the same way that the metabolite (i.e., p-nitrophenolate) undergoes fragmentation to provide daughter ions, as depicted below:

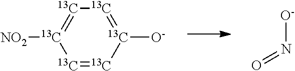

Thus, the internal standard will provide daughter ions having an m/z of 46. However, unlike the daughter ion having an m/z of 46 that is derived from the p-nitrophenol formed by the alkaline phosphatase (which is derived from a parent ion having an m/z ratio of 138), the daughter ion is derived from a parent ion having an m/z ratio of 144.

When the sample includes p-nitrophenol labeled with ¹³C at each of the carbon atoms as an internal standard, the mass spectrometer is configured so that the first quadrupole separates parent ions having an m/z ratio of 138 (p-nitrophenolate formed from the substrate) and 144 (p-nitrophenol labeled with ¹³C at each of the carbon atoms formed from the internal standard) and the third quadrupole separates daughter ions having an m/z ratio of 46 (daughter ions derived from p-nitrophenol formed from the substrate and daughter ions derived from p-nitrophenol labeled with ¹³C at each of the carbon atoms formed from the internal standard). The detector is configured to record the intensity of ions having an m/z ratio of 46 derived from parent ion having an m/z ratio of 138 and an m/z ratio of 46 derived from parent ion having an m/z ratio of 144, i.e., 46/138 and 46/144 peaks.

The internal standard is added to the sample in a known amount. The ratio of the intensity of the 46/138 peak to the intensity of the 46/144 peak is used to determine the amount of p-nitrophenol formed by the alkaline phosphatase catalyzed conversion of p-nitrophenyl phosphate to p-nitrophenol.

The alkaline phosphatase activity can be quantified by comparing the ratio of the intensity of the 46/138 peak to the intensity of the 46/144 peak obtained from the sample with the ratio of the intensity of the 46/138 peak to the intensity of the 46/144 peak obtained from a positive control that includes a known amount of alkaline phosphatase of a known activity (e.g., alkaline phosphatase, from bovine intestinal mucosa, commercially available from Sigma Aldrich of St. Louis, MO) that is treated in the same way that the sample is treated.

For example, if the positive control has an alkaline phosphatase activity of 25 units/liter and provides a value of 100 for the ratio of the intensity of the 46/138 peak to the intensity of the 46/144 peak and the sample provides a value of 50 for the ratio of the intensity of the 46/138 peak to the intensity of the 46/144 peak, the alkaline phosphatase activity in the sample would be 12.5 units/liter.

Thus, in one embodiment, the method for assaying alkaline phosphatase activity in a sample comprises:
(i) providing a sample suspected of containing alkaline phosphatase;
(ii) contacting the sample with p-nitrophenyl phosphate to provide an assay mixture;
(iii) allowing the assay mixture to incubate at a temperature for a period of time;
(iv) quenching the assay mixture;
(v) passing the quenched assay mixture through an HPLC column to provide an eluant containing components of the assay mixture,
(vi) subjecting at least a portion of the eluant to ionization in a mass spectrometer in the negative ion mode to provide a plurality of parent ions;
(vii) separating parent ions having an m/z ratio of 138 from the plurality of parent ions;
(viii) fragmenting the parent ions having an m/z ratio of 138 to provide a plurality of daughter ions;
(ix) separating daughter ions that have an m/z ratio of 46 from the plurality of daughter ions; and
(x) detecting the intensity daughter ions that have an m/z ratio of 46.

In one embodiment, step (ii) involves contacting the sample with p-nitrophenyl phosphate, a zinc salt, a magnesium salt, and HEDTA to provide the assay mixture. In one embodiment, step (ii) involves contacting the sample with p-nitrophenyl phosphate, zinc sulfate, magnesium acetate, and HEDTA to provide the assay mixture.

In one embodiment, the assay further comprises adding p-nitrophenol labeled with ¹³C at each of the carbon atoms to the sample as an internal standard. When the assay comprises adding p-nitrophenol labeled with ¹³C at each of the carbon atoms as an internal standard, the assay involves separating parent ions having an m/z ratio of 138 and 144 from the plurality of parent ions and detecting the daughter ions having an m/z ratio of 46 derived from a parent ion having an m/z ratio of 138 and an m/z ratio of 46 derived from a parent ion having an m/z ratio of 144, i.e., detecting the 46/138 and 46/144 peaks.

In one embodiment, the alkaline phosphatase activity can be quantified by creating a standard curve prepared by adding a fixed concentration of p-nitrophenol labeled with ¹³C at each of the carbon atoms (i.e., the internal standard) to a series of solutions containing p-nitrophenol (i.e., ALP product) of different concentrations, determining the intensity of the 46/138 peak relative to the intensity of the 46/144 peak for each solution, and plotting the intensity of the 46/138 peak relative to the intensity of the 46/144 vs. concentration of the ALP product. The sample is prepared so that it includes the same concentration of internal standard. The intensity of the 46/138 peak relative to the intensity of the 46/144 from the sample is determined and the concentration of the ALP product in the sample is obtained from the standard curve. The enzyme activity, typically reported in “activity units per liter” (U/L, where U=μmol product/min), is determined according to the following equation:

$$\frac{( \frac{( μ g / mL \times 1000 )}{139.11 g / mol} )}{t} = U / L$$

wherein concentration (obtained from the standard curve) is in μg/mL and t is the incubation time in minutes.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the quenched assay mixture is passed through an HPLC column using HPLC conditions A.

### Assay for Glucose

The method for assaying for glucose levels in a sample comprises:
(i) providing a sample suspected of containing glucose;
(ii) adding isotopically labelled glucose to the sample, wherein the isotopically labelled glucose contains an isotopic label, to provide an assay mixture;
(iii) using mass spectrometry operated in the negative ion mode, to generate and detect (a) an ion formed from the glucose and (b) an ion formed from the isotopically labelled glucose, wherein the ion formed from the isotopically labelled glucose comprises the isotopic label.

In one embodiment, the ion formed from the glucose is a daughter ion and an ion formed from the isotopically labelled glucose is a daughter ion.

The pH of the assay mixture can vary over a wide range. In one embodiment, the assay mixture has a pH of between about 6.0 and about 9.0, preferable between about 7.0 and about 8.0, so that the assay can be conducted simultaneously with the assay for alanine transaminase activity and/or aspartate transaminase activity. In one embodiment, the pH is about 7.4.

In one embodiment, the assay mixture has a pH of between about 8.0 and about 12.0, preferable between about 9.0 and about 11.0, so that the assay can be conducted simultaneously with the assay for alkaline phosphatase activity. In one embodiment, the pH is about 10.2.

Suitable buffers for maintaining the pH include, but are not limited to, those described above.

In one embodiment, the isotopically labelled glucose is (2R,3R,4S,5S,6R)-6-(hydroxymethyl-¹³C) tetrahydro-2H-pyran-2,3,4,5-tetraol-2,3,4,5,6-¹³C, i.e.:

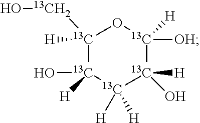

hereinafter referred to as “¹³C-labeled glucose.”

¹³C-labeled glucose is commercially available from Cambridge Isotope Laboratories, Inc. of Tewksbury, MA and Sigma Aldrich Inc. of St. Louis, MO.

In one embodiment, using ¹³C-labeled glucose as the isotopically labelled glucose, the method comprises:
(i) providing a sample suspected of containing glucose;
(ii) adding ¹³C-labeled glucose to the sample, to provide an assay mixture;
(iii) subjecting at least a portion of the assay mixture to ionization in a mass spectrometer in the negative ion mode to provide a plurality of parent ions;
(ii) separating the parent ions having an m/z ratio of 179 and an m/z ratio of 185 from the plurality of parent ions;
(iii) fragmenting the parent ions having an m/z ratio of 179 and an m/z ratio of 185 to provide a plurality of daughter ions;
(iv) separating daughter ions that have an m/z ratio of 119 and an m/z ratio of 123 from the plurality of daughter ions; and
(v) detecting the intensity of the daughter ions that have an m/z ratio of 119 and an m/z ratio of 123.

Without wishing to be bound by theory it is believed that the glucose molecules form a parent ion having the structure:

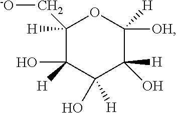

which has an m/z ratio of 179, that then fragments to a daughter ion having the structure:

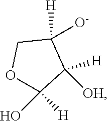

which has an m/z ratio of 119.

Without wishing to be bound by theory it is believed that the ¹³C-labeled glucose molecules form a parent ion having the structure:

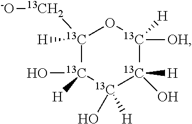

which has an m/z ratio of 185, that fragments to a daughter ion having the structure:

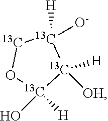

which has an m/z ratio of 123.

The assay can be performed using a triple quadrupole mass spectrometer wherein the first quadrupole separates the parent ions having an m/z ratio of 179 and an m/z ratio of 185 from the other parent ions; the second quadrupole is a collision chamber that fragments the parent ions having an m/z ratio of 179 and an m/z ratio of 185 to provide daughter ions, and the third quadrupole separates daughter ions that have an m/z ratio of 119 and an m/z ratio of 123 from other daughter ions.

The ¹³C-labeled glucose is added in a known amount. The ratio of the intensity of the mass spectrum peak having an m/z ratio of 119 to the intensity of the mass spectrum peak having an m/z ratio of 123, i.e., the ratio of the intensity of the 119/179 peak to the intensity of the 123/185 peak, is used to determine the amount of glucose in the sample.

For example if the intensity of the 119/179 peak is 200 and the intensity of the 123/185 peak is 100, the amount of glucose in the sample is twice the amount of ¹³C labeled glucose that was added to the sample.

In one embodiment, the glucose level is quantified by creating a standard curve prepared by adding a fixed concentration of ¹³C-labeled glucose (i.e., the internal standard) to a series of solutions containing glucose of different concentrations, determining the intensity of the 119/179 peak relative to the intensity of the 123/185 peak for each solution, and plotting the intensity of the 119/179 peak relative to the intensity of the 123/185 peak vs. the concentration of glucose. The sample is prepared so that it includes the same concentration of internal standard. The intensity of the 119/179 peak relative to the intensity of the 123/185 peak from the sample is determined and the concentration of glucose in the sample is obtained from the standard curve.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the assay mixture is passed through an HPLC column to provide an eluant containing components of the assay mixture and at least a portion of the eluant is ionized in the mass spectrometer operated in the negative ion mode to detect the ion formed from the glucose and the ion formed from the isotopically labelled glucose.

In one embodiment, the assay mixture is passed through an HPLC column using HPLC conditions A.

An advantage of the method for assaying glucose is that it can be performed simultaneously with the assay for alanine transaminase activity and/or aspartate transaminase activity. When the method involves simultaneously assaying for glucose, alanine transaminase activity, and/or aspartate transaminase activity, the sample is adjusted to a pH of between about 6.0 and about 9.0, preferably between about 7.0 and about 8.0. In one embodiment, the pH is about 7.4. The incubation conditions and quenching step associated with the assay for alanine transaminase activity and/or aspartate transaminase activity do not interfere with the assay for glucose.

As an example, the assay for glucose is performed simultaneously with the assay for alanine transaminase activity. In such a simultaneous assay the mass spectrometer can be configured to detect the intensity of the 119/179 peak (i.e., the daughter ion derived from glucose), the intensity of the 123/185 peak (i.e., the daughter ion derived from the glucose internal standard, i.e., ¹³C-labeled glucose), the intensity of the 43/88 peak (i.e., the daughter ion derived from the pyruvate formed by the action of alanine transaminase on the substrate (the ALT product), i.e., alanine labeled with ¹³C in the 1-position), and the intensity of the 44/89 peak (i.e., the daughter ion from derived from the pyruvate internal standard, i.e., pyruvate labeled with a ¹³C at the 1- and 2-positions).

Another advantage of the method for assaying glucose is that it can be performed simultaneously with the assay for alkaline phosphatase activity. When the method involves simultaneously assaying for alkaline phosphatase activity and glucose levels, the sample is adjusted to a pH of between about 9.0 and about 12.0, preferable between about 9.5 and about 11.0. In one embodiment, the pH is about 10.2. The incubation conditions and quenching step associated with the assay for alkaline phosphatase activity do not interfere with the assay for glucose.

In the method for simultaneously assaying alkaline phosphatase activity and glucose levels the mass spectrometer can be configured to detect the intensity of the 119/179 peak (i.e., the daughter ion derived from glucose), the intensity of the 123/185 peak (i.e., the daughter ion derived from the glucose internal standard, i.e., ¹³C-labeled glucose), the intensity of the 46/138 peak (i.e., the daughter ion from derived from p-nitrophenol formed by the action of alkaline phosphatase on the substrate, i.e., the ALP product), and the intensity of the 44/144 peak (i.e., the daughter ion from derived from the internal standard, i.e., p-nitrophenol labeled with ¹³C at each of the carbon atoms).

### Assay for Urea

The method for assaying for urea levels in a sample comprises:
(i) providing a sample suspected of containing urea;
(ii) adding isotopically labelled urea to the sample, wherein the isotopically labelled urea contains an isotopic label, to provide an assay mixture;
(iii) using mass spectrometry in the positive ion mode to generate and detect (a) an ion formed from the urea and (b) an ion formed from the isotopically labelled urea, wherein the ion formed from the isotopically labelled urea comprises the isotopic label.

In one embodiment, the ion formed from the urea is a daughter ion and an ion formed from the isotopically labelled urea is a daughter ion.

The pH of the assay mixture can vary over a wide range. In one embodiment, the assay mixture has a pH of between about 6.0 and about 9.0, preferable between about 7.0 and about 8.0, so that the assay can be conducted simultaneously with the assay for alanine transaminase activity and/or aspartate transaminase activity. In one embodiment, the pH is about 7.4.

In one embodiment, the assay mixture has a pH of between about 8.0 and about 12.0, preferable between about 9.0 and about 11.0, so that the assay can be conducted simultaneously with the assay for alkaline phosphatase activity. In one embodiment, the pH is about 10.2.

Suitable buffers for maintaining the pH include, but are not limited to, those described above.

In one embodiment, the isotopically labelled urea is urea labeled with ¹³C and ¹⁵N, i.e.:

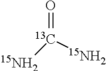

Urea labeled with ¹³C and ¹⁵N is commercially available from Sigma Aldrich Inc. of St. Louis, MO and Cambridge Isotope Laboratories, Inc. of Tewksbury, MA.

In one embodiment, using urea labeled with ¹³C and ¹⁵N as the isotopically labelled urea, the method comprises:
(i) providing a sample suspected of containing urea;
(ii) adding urea labeled with ¹³C and ¹⁵N to the sample, to provide an assay mixture;
(iii) subjecting at least a portion of the assay mixture to ionization in a mass spectrometer operated in the positive ion mode to provide a plurality of parent ions;
(iv) separating the parent ions having an m/z ratio of 61 and an m/z ratio of 64 from the plurality of parent ions;
(v) fragmenting the parent ions having an m/z ratio of 61 and an m/z ratio of 64 to provide a plurality of daughter ions;
(vi) separating daughter ions that have an m/z ratio of 44 and an m/z ratio of 46 from the plurality of daughter ions; and
(vii) detecting the intensity of the daughter ions that have an m/z ratio of 44 and an m/z ratio of 46.

Without wishing to be bound by theory it is believed that the urea molecules form a parent ion having the structure:

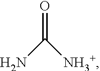

which has an m/z ratio of 61, that then fragments to a daughter ion having the structure:

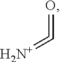

which has an m/z ratio of 44.

Without wishing to be bound by theory it is believed that the urea labeled with ¹³C and ¹⁵N molecules form a parent ion having the structure:

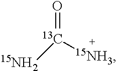

which has an m/z ratio of 64, that fragments to a daughter ion having the structure:

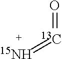

which has an m/z ratio of 46.

The assay can be performed using a triple quadrupole mass spectrometer wherein the first quadrupole separates the parent ions having an m/z ratio of 61 and an m/z ratio of 64 from other parent ions; the second quadrupole is a collision chamber that fragments the parent ions having an m/z ratio of 61 and an m/z ratio of 64 to provide daughter ions, and the third quadrupole separates daughter ions that have an m/z ratio of 44 and an m/z ratio of 46 from other daughter ions.

The urea labeled with ¹³C and ¹⁵N molecules is added in a known amount. The ratio of the intensity of the mass spectrum peak having an m/z ratio of 44 to the intensity of the mass spectrum peak having an m/z of 46, i.e., the ratio of the intensity of the 44/61 peak to the intensity of the 46/64 peak is used to determine the amount of urea in the sample.

For example, if the intensity of the 44/61 peak is 100 and the intensity of the 46/64 peak is 200, the amount of urea in the sample is one-half the amount of urea labeled with ¹³C and ¹⁵N that was added to the sample.

In one embodiment, the urea level is quantified by creating a standard curve prepared by adding a fixed concentration of urea labeled with ¹³C and ¹⁵N molecules (i.e., the internal standard) to a series of solutions containing urea of different concentrations, determining the intensity of the 44/61 peak relative to the intensity of the 46/64 peak for each solution, and plotting the intensity of the 44/61 peak relative to the intensity of the 46/64 peak vs. the concentration of urea. The sample is prepared so that it includes the same concentration of internal standard. The intensity of the 44/61 peak relative to the intensity of the 46/64 peak from the sample is determined and the concentration of urea in the sample is obtained from the standard curve.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the assay mixture is passed through an HPLC column to provide an eluant containing components of the assay mixture and at least a portion of the eluant is ionized in the mass spectrometer operated in the positive ion mode to detect the ion formed from the urea and the ion formed from the isotopically labelled urea.

In one embodiment, the assay mixture is passed through an HPLC column using HPLC conditions B.

An advantage of the method for assaying urea is that it can be performed simultaneously with the assay for alanine transaminase activity and/or aspartate transaminase activity. When the method involves simultaneously assaying for urea, alanine transaminase activity, and/or aspartate transaminase activity, the sample is adjusted to a pH of between about 6.0 and about 9.0, preferably between about 7.0 and about 8.0. In one embodiment, the pH is about 7.4. The incubation conditions and quenching step associated with the assay for alanine transaminase activity and aspartate transaminase activity do not interfere with the assay for urea.

As an example, the assay for urea is performed simultaneously with the assay for alanine transaminase activity (but using two separate modes of the mass spectrometer, i.e., positive ion mode and negative ion mode). In such a simultaneous assay the mass spectrometer can be configured to detect the intensity of the 44/61 peak (i.e., the daughter ion derived from urea), the intensity of the 46/64 peak (i.e., the daughter ion derived from the urea internal standard, i.e., urea labeled with ¹³C and ¹⁵N molecules), the intensity of the 43/88 peak (i.e., the daughter ion derived from the pyruvate formed by the action of alanine transaminase on the substrate (i.e., the ALT product), i.e., alanine labeled with ¹³C in the 1-position), and the intensity of the 44/89 peak (i.e., the daughter ion from derived from the pyruvate internal standard, i.e., pyruvate labeled with a ¹³C at the 1- and 2-positions).

Another advantage of the method for assaying urea is that it can be performed simultaneously with the assay for alkaline phosphatase activity. When the method involves simultaneously assaying for alkaline phosphatase activity and urea levels, the sample is adjusted to a pH of between about 9.0 and about 12.0, preferable between about 9.5 and about 11.0. In one embodiment, the pH is about 10.2. The incubation conditions and quenching step associated with the assay for alkaline phosphatase activity do not interfere with the assay for urea.

In the method for simultaneously assaying alkaline phosphatase activity and urea levels the mass spectrometer can be configured to detect the intensity of the 44/61 peak (i.e., the daughter ion derived from urea), the intensity of the 46/64 peak (i.e., the daughter ion derived from the urea internal standard, i.e., urea labeled with ¹³C and ¹⁵N molecules), the intensity of the 46/138 peak (i.e., the daughter ion from derived from p-nitrophenol formed by the action of alkaline phosphatase on the substrate, i.e., the ALP product), and the intensity of the 44/144 peak (i.e., the daughter ion from derived from the internal standard, i.e., p-nitrophenol labeled with ¹³C at each of the carbon atoms).

### Assay for Creatinine

The method for assaying for creatinine levels in a sample comprises:
(i) providing a sample suspected of containing creatinine;
(ii) adding isotopically labelled creatinine to the sample, wherein the isotopically labelled creatinine contains an isotopic label, to provide an assay mixture;
(iii) using mass spectrometry in the positive ion mode to generate and detect (a) an ion formed from the creatinine and (b) an ion formed from the isotopically labelled creatinine, wherein the ion formed from the isotopically labelled creatinine comprises the isotopic label.

In one embodiment, the ion formed from the creatinine is a daughter ion and an ion formed from the isotopically labelled creatinine is a daughter ion.

The pH of the assay mixture can vary over a wide range. In one embodiment, the assay mixture has a pH of between about 6.0 and about 9.0, preferable between about 7.0 and about 8.0, so that the assay can be conducted simultaneously with the assay for alanine transaminase activity and/or aspartate transaminase activity. In one embodiment, the pH is about 7.4.

In one embodiment, the assay mixture has a pH of between about 8.0 and about 12.0, preferable between about 9.0 and about 11.0, so that the assay can be conducted simultaneously with the assay for alkaline phosphatase activity. In one embodiment, the pH is about 10.2.

Suitable buffers for maintaining the pH include, but are not limited to, those described above.

In one embodiment, the isotopically labelled creatinine is creatinine with a deuterated methyl group (i.e., 2-imino-1-(methyl-d₃)-2,5-dihydro-1H-imidazol-4-ol), i.e.:

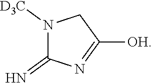 [SMILES: [2H]C([2H])([2H])N1CC(O)=NC1=N]

Creatinine with a deuterated methyl group is commercially available from Cambridge Isotope Laboratories, Inc. of Tewksbury, MA.

In one embodiment, using creatinine with a deuterated methyl group as the isotopically labelled creatinine, the method comprises:
(i) providing a sample suspected of containing creatinine;
(ii) adding creatinine with a deuterated methyl group to the sample, to provide an assay mixture;
(iii) subjecting at least a portion of the assay mixture to ionization in a mass spectrometer in the positive ion mode to provide a plurality of parent ions;
(ii) separating the parent ions having an m/z ratio of 114 and an m/z ratio of 117 from the plurality of parent ions;
(iv) fragmenting the parent ions having an m/z ratio of 114 and an m/z ratio of 117 to provide a plurality of daughter ions;
(iv) separating daughter ions that have an m/z ratio of 44 and an m/z ratio of 47 from the plurality of daughter ions or separating daughter ions having an m/z ratio of 86 and 89 from the plurality of daughter ions; and
(v) detecting the intensity of the daughter ions that have an m/z ratio of 44 and an m/z ratio of 47 or detecting the intensity of the daughter ions that have an m/z ratio of 86 and an m/z ratio of 89.

Without wishing to be bound by theory, it is believed that the creatinine molecules form a parent ion having the structure:

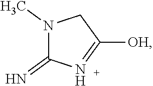

which has an m/z ratio of 114, that then fragments to a daughter ion having the structure:

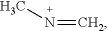

which has an m/z ratio of 44.

Without wishing to be bound by theory, it is believed that the creatinine parent ion also fragments into a daughter ion having the structure:

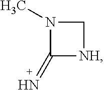

which has an m/z ratio of 86.

Without wishing to be bound by theory, it is believed that the creatinine molecules with a deuterated methyl group form a parent ion having the structure:

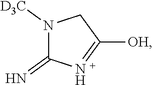 [SMILES: [2H]C([2H])([2H])N1CC(O)=NC1=N]

which has an m/z ratio of 117, that fragments to a daughter ion having the structure:

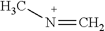

which has an m/z ratio of 47.

Without wishing to be bound by theory, it is believed that the creatinine molecules with a deuterated methyl group also fragments to a daughter ion having the structure:

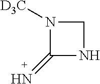

which has an m/z ratio of 89.

The assay can be performed using a triple quadrupole mass spectrometer wherein the first quadrupole separates the parent ions having an m/z ratio of 114 and an m/z ratio of 117 from other parent ions; the second quadrupole is a collision chamber that fragments the parent ions having an m/z ratio of 114 and an m/z ratio of 117 to provide daughter ions, and the third quadrupole separates daughter ions that have an m/z ratio of 44 and an m/z ratio of 47 from other daughter ions or separates daughter ions that have an m/z ratio of 86 and an m/z ratio of 89 from other daughter ions.

The creatinine with a deuterated methyl group is added in a known amount. The ratio of the intensity of the mass spectrum peak having an m/z ratio of 44 relative to the intensity of the mass spectrum peak having an m/z of 47, i.e., the ratio of the intensity 44/114 peak to the 47/117 peak, is used to determine the amount of creatinine in the sample.

For example, if the intensity of the 44/114 peak is 100 and the intensity of the 47/117 peak is 200, the amount of creatinine in the sample is one-half the amount of creatinine with a deuterated methyl group that was added to the sample.

Alternatively, the ratio of the intensity of the mass spectrum peak having an m/z ratio of 86 relative to the intensity of the mass spectrum peak having an m/z of 89, i.e., the ratio of the intensity 86/114 peak to the 89/117 peak, is used to determine the amount of creatinine in the sample.

For example if the intensity of the 86/114 peak is 100 and the intensity of the 89/117 peak is 200, the amount of creatinine in the sample is one-half the amount of creatinine with a deuterated methyl group that was added to the sample.

In one embodiment, the creatinine level is quantified by creating a standard curve prepared by adding a fixed concentration of creatine with a deuterated methyl group (i.e., the internal standard) to a series of solutions containing creatinine of different concentrations, determining the intensity of the 44/114 peak relative to the intensity of the 47/117 peak for each solution, and plotting the intensity of the 44/114 peak relative to the intensity of the 47/117 peak vs. the concentration of creatinine. The sample is prepared so that it includes the same concentration of internal standard. The intensity of the 44/114 peak relative to the intensity of the 47/117 peak from the sample is determined and the concentration of creatinine in the sample is obtained from the standard curve.

In one embodiment, the creatinine level is quantified by creating a standard curve prepared by adding a fixed concentration of creatine with a deuterated methyl group (i.e., the internal standard) to a series of solutions containing creatinine of different concentrations, determining the intensity of the 86/114 peak relative to the intensity of the 89/117 peak for each solution, and plotting the intensity of the 86/114 peak relative to the intensity of the 89/117 vs. the concentration of creatinine. The sample is prepared so that it includes the same concentration of internal standard. The intensity of the 86/114 peak relative to the intensity of the 89/117 from the sample is determined and the concentration of creatinine in the sample is obtained from the standard curve.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the assay mixture is passed through an HPLC column to provide an eluant containing components of the assay mixture and at least a portion of the eluant is ionized in the mass spectrometer operated in the positive ion mode to detect the ion formed from the creatinine and the ion formed from the isotopically labelled creatinine.

In one embodiment, the assay mixture is passed through an HPLC column using HPLC conditions B.

An advantage of the method for assaying creatinine is that it can be performed simultaneously with the assay for alanine transaminase activity and/or aspartate transaminase activity. When the method involves simultaneously assaying for creatinine, alanine transaminase activity, and/or aspartate transaminase activity, the sample is adjusted to a pH of between about 6.0 and about 9.0, preferably between about 7.0 and about 8.0. In one embodiment, the pH is about 7.4. The incubation conditions and quenching step associated with the assay for alanine transaminase activity and aspartate transaminase activity do not interfere with the assay for creatinine.

As an example, the assay for creatinine is performed simultaneously with the assay for alanine transaminase activity (but using two separate modes of the mass spectrometer, i.e., positive ion mode and negative ion mode). In such a simultaneous assay the mass spectrometer could be configured to detect the intensity of the 44/114 (or 86/114) peak (i.e., the daughter ion derived from creatinine), the intensity of the 47/117 (or 89/117) peak (i.e., the daughter ion derived from the creatinine internal standard, i.e., creatinine with a deuterated methyl group), the intensity of the 43/88 peak (i.e., the daughter ion derived from the pyruvate formed by the action of alanine transaminase on the substrate (the ALT product), i.e., alanine labeled with ¹³C in the 1-position), and the intensity of the 44/89 peak (i.e., the daughter ion from derived from the pyruvate internal standard, i.e., pyruvate labeled with a ¹³C at the 1- and 2-positions).

Another advantage of the method for assaying creatinine is that it can be performed simultaneously with the assay for alkaline phosphatase activity. When the method involves simultaneously assaying for alkaline phosphatase activity and creatinine levels, the sample is adjusted to a pH of between about 9.0 and about 12.0, preferable between about 9.5 and about 11.0. In one embodiment, the pH is about 10.2. The incubation conditions and quenching step associated with the assay for alkaline phosphatase activity do not interfere with the assay for creatinine.

In the method for simultaneously assaying alkaline phosphatase activity and creatinine levels the mass spectrometer can be configured to detect the intensity of the 44/114 (or 86/114) peak (i.e., the daughter ion derived from creatinine), the intensity of the 47/117 (or 89/117) peak (i.e., the daughter ion derived from the creatinine internal standard, i.e., creatinine with a deuterated methyl group), the intensity of the 46/138 peak (i.e., the daughter ion from derived from p-nitrophenol formed by the action of alkaline phosphatase on the substrate, i.e., the ALP product), and the intensity of the 44/144 peak (i.e., the daughter ion from derived from the internal standard, i.e., p-nitrophenol labeled with ¹³C at each of the carbon atoms).

### Assay for ADMA

The method for assaying for ADMA levels in a sample comprises:
(i) providing a sample suspected of containing ADMA;
(ii) adding isotopically labelled ADMA to the sample, wherein the isotopically labelled ADMA contains an isotopic label, to provide an assay mixture;
(iii) using mass spectrometry in the positive ion mode to generate and detect (a) an ion formed from the ADMA and (b) an ion formed from the isotopically labelled ADMA, wherein the ion formed from the isotopically labelled ADMA comprises the isotopic label.

In one embodiment, the ion formed from the ADMA is a daughter ion and an ion formed from the isotopically labelled ADMA is a daughter ion.

The pH of the assay mixture can vary over a wide range. In one embodiment, the assay mixture has a pH of between about 6.0 and about 9.0, preferable between about 7.0 and about 8.0, so that the assay can be conducted simultaneously with the assay for alanine transaminase activity and/or aspartate transaminase activity. In one embodiment, the pH is about 7.4.

In one embodiment, the assay mixture has a pH of between about 8.0 and about 12.0, preferable between about 9.0 and about 11.0, so that the assay can be conducted simultaneously with the assay for alkaline phosphatase activity. In one embodiment, the pH is about 10.2.

Suitable buffers for maintaining the pH include, but are not limited to, those described above.

In one embodiment, the isotopically labelled ADMA is deuterated ADMA, i.e.:

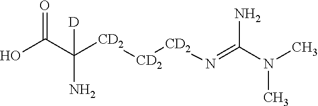

deuterated ADMA is commercially available from Cambridge Isotope Laboratories, Inc. of Tewksbury, MA.

In one embodiment, using deuterated ADMA as the isotopically labelled ADMA, the method comprises:
(i) providing a sample suspected of containing ADMA;
(ii) adding deuterated ADMA to the sample, to provide an assay mixture;
(iii) subjecting at least a portion of the assay mixture to ionization in a mass spectrometer in the positive ion mode to provide a plurality of parent ions;
(iv) separating the parent ions having an m/z ratio of 203 and an m/z ratio of 210 from the plurality of parent ions;
(v) fragmenting the parent ions having an m/z ratio of 203 and an m/z ratio of 210 to provide a plurality of daughter ions;
(vi) separating daughter ions that have an m/z ratio of 46 from the plurality of daughter ions; and
(vii) detecting the intensity of the daughter ions that have an m/z ratio of 46.

Without wishing to be bound by theory, it is believed that the ADMA molecules form a parent ion having the structure:

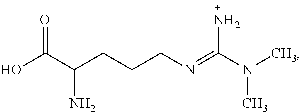

which has an m/z ratio of 203, that then fragments to a daughter ion having the structure:

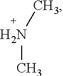

which has an m/z ratio of 46.

Without wishing to be bound by theory, it is believed that the deuterated ADMA molecules form a parent ion having the structure:

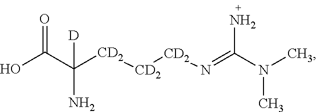

which has an m/z ratio of 210, that also fragments to a daughter ion having the structure:

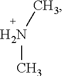

which has an m/z ratio of 46.

The assay can be performed using a triple quadrupole mass spectrometer wherein the first quadrupole separates the parent ions having an m/z ratio of 203 and an m/z ratio of 210 from other parent ions; the second quadrupole is a collision chamber that fragments the parent ions having an m/z ratio of 203 and an m/z ratio of 210 to provide daughter ions, and the third quadrupole separates daughter ions that have an m/z ratio of 46 from other daughter ions.

The deuterated ADMA is added in a known amount. The ratio of the intensity of the mass spectrum peak having an m/z ratio of 46 derived from a parent ion having an m/z ratio of 203 to the intensity of the mass spectrum peak having an m/z ratio of 46 derived from a parent ion having an m/z ratio 210, i.e., the ratio of the intensity of the 46/203 peak to the intensity of the 46/210 peak, is used to determine the amount of ADMA in the sample.

For example if the intensity of the 46/203 peak is 100 and the intensity of the 46/210 peak is 200, the amount of ADMA in the sample is one-half the amount of deuterated ADMA that was added to the sample.

In one embodiment, the ADMA level is quantified by creating a standard curve prepared by adding a fixed concentration of deuterated ADMA (i.e., the internal standard) to a series of solutions containing ADMA of different concentrations, determining the intensity of the 46/203 peak relative to the intensity of the 46/210 peak for each solution, and plotting the intensity of the 46/203 peak relative to the intensity of the 46/210 peak vs. the concentration of ADMA. The sample is prepared so that it includes the same concentration of internal standard. The intensity of the 46/203 peak relative to the intensity of the 46/210 peak from the sample is determined and the concentration of ADMA in the sample is obtained from the standard curve.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the assay mixture is passed through an HPLC column to provide an eluant containing components of the assay mixture and at least a portion of the eluant is ionized in the mass spectrometer operated in the positive ion mode to detect the ion formed from the ADMA and the ion formed from the isotopically labelled ADMA.

In one embodiment, the assay mixture is passed through an HPLC column using HPLC conditions B.

An advantage of the method for assaying ADMA is that it can be performed simultaneously with the assay for alanine transaminase activity and/or aspartate transaminase activity. When the method involves simultaneously assaying for ADMA, alanine transaminase activity, and/or aspartate transaminase activity, the sample is adjusted to a pH of between about 6.0 and about 9.0, preferably between about 7.0 and about 8.0. In one embodiment, the pH is about 7.4. The incubation conditions and quenching step associated with the assay for alanine transaminase activity and aspartate transaminase activity do not interfere with the assay for ADMA.

As an example, the assay for ADMA is performed simultaneously with the assay for alanine transaminase activity (but using two separate modes of the mass spectrometer, i.e., positive ion mode and negative ion mode). In such a simultaneous assay the mass spectrometer could be configured to detect the intensity of the 46/203 peak (i.e., the daughter ion derived from ADMA), the intensity of the 46/210 peak (i.e., the daughter ion derived from the ADMA internal standard, i.e., deuterated ADMA), the intensity of the 43/88 peak (i.e., the daughter ion derived from the pyruvate formed by the action of alanine transaminase on the substrate (i.e., the ALT product), i.e., alanine labeled with ¹³C in the 1-position), and the intensity of the 44/89 peak (i.e., the daughter ion from derived from the pyruvate internal standard, i.e., pyruvate labeled with a ¹³C at the 1- and 2-positions).

Another advantage of the method for assaying ADMA is that it can be performed simultaneously with the assay for alkaline phosphatase activity. When the method involves simultaneously assaying for alkaline phosphatase activity and ADMA levels, the sample is adjusted to a pH of between about 9.0 and about 12.0, preferable between about 9.5 and about 11.0. In one embodiment, the pH is about 10.2. The incubation conditions and quenching step associated with the assay for alkaline phosphatase activity do not interfere with the assay for ADMA.

In the method for simultaneously assaying alkaline phosphatase activity and ADMA levels the mass spectrometer can be configured to detect the intensity of the 46/203 peak (i.e., the daughter ion derived from ADMA), the intensity of the 46/210 peak (i.e., the daughter ion derived from the ADMA internal standard, i.e., deuterated ADMA), the intensity of the 46/138 peak (i.e., the daughter ion from derived from p-nitrophenol formed by the action of alkaline phosphatase on the substrate, i.e., the ALP product), and the intensity of the 44/144 peak (i.e., the daughter ion from derived from the internal standard, i.e., p-nitrophenol labeled with ¹³C at each of the carbon atoms).

### Assay for SDMA

The method for assaying for SDMA levels in a sample comprises:
(i) providing a sample suspected of containing SDMA;
(ii) adding isotopically labelled SDMA to the sample, wherein the isotopically labeled SDMA contains an isotopic label, to provide an assay mixture;
(iii) using mass spectrometry in the positive ion mode to generate and detect (a) an ion formed from the SDMA and (b) an ion formed from the isotopically labelled SDMA, wherein the ion formed from the isotopically labelled SDMA comprises the isotopic label.

In one embodiment, the ion formed from the SDMA is a daughter ion and an ion formed from the isotopically labelled SDMA is a daughter ion.

The pH of the assay mixture can vary over a wide range. In one embodiment, the assay mixture has a pH of between about 6.0 and about 9.0, preferable between about 7.0 and about 8.0, so that the assay can be conducted simultaneously with the assay for alanine transaminase activity and/or aspartate transaminase activity. In one embodiment, the pH is about 7.4.

In one embodiment, the assay mixture has a pH of between about 8.0 and about 12.0, preferable between about 9.0 and about 11.0, so that the assay can be conducted simultaneously with the assay for alkaline phosphatase activity. In one embodiment, the pH is about 10.2.

Suitable buffers for maintaining the pH include, but are not limited to, those described above.

In one embodiment, the isotopically labelled SDMA is deuterated SDMA, i.e.:

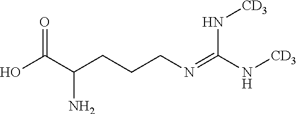 [SMILES: [2H]C([2H])([2H])NC(=NCCCC(N)C(=O)O)NC([2H])([2H])[2H]]

deuterated SDMA is commercially available from Toronto Research Chemicals of North York, ON, Canada.

In one embodiment, using deuterated SDMA as the isotopically labelled SDMA, the method comprises:
(i) providing a sample suspected of containing SDMA;
(ii) adding deuterated SDMA to the sample, to provide an assay mixture;
(iii) subjecting at least a portion of the assay mixture to ionization in a mass spectrometer in the positive ion mode to provide a plurality of parent ions;
(iv) separating the parent ions having an m/z ratio of 203 and an m/z ratio of 209 from the plurality of parent ions;
(v) fragmenting the ions having an m/z ratio of 203 and an m/z ratio of 209 to provide a plurality of daughter ions;
(vi) separating daughter ions that have an m/z ratio of 172 and an m/z ratio of 175 from the plurality of daughter ions; and
(vii) detecting the intensity of the daughter ions that have an m/z ratio of 172 and an m/z ratio of 175.

Without wishing to be bound by theory, it is believed that the SDMA molecules form a parent ion having the structure:

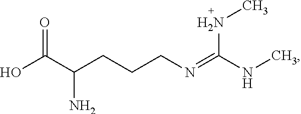

which has an m/z ratio of 203, that then fragments to a daughter ion having the structure:

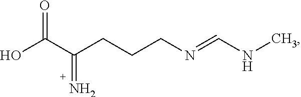 [SMILES: CN/C=N/CCCC(=N)C(=O)O]

which has an m/z ratio of 172.

Without wishing to be bound by theory, it is believed that the deuterated SDMA molecules form a parent ion having the structure:

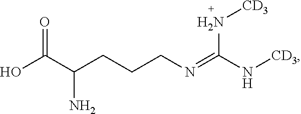 [SMILES: [2H]C([2H])([2H])NC(=NCCCC(N)C(=O)O)NC([2H])([2H])[2H]]

which has an m/z ratio of 209, that fragments to a daughter ion having the structure:

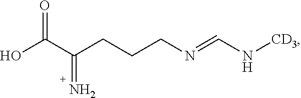 [SMILES: [2H]C([2H])([2H])N/C=N/CCCC(=N)C(=O)O]

which has an m/z ratio of 175.

The assay can be performed using a triple quadrupole mass spectrometer wherein the first quadrupole separates the parent ions having an m/z ratio of 203 and an m/z ratio of 209 from other parent ions; the second quadrupole is a collision chamber that fragments the parent ions having an m/z ratio of 203 and an m/z ratio of 209 to provide daughter ions, and the third quadrupole separates daughter ions that have an m/z ratio of 172 and an m/z ratio of 175 from other daughter ions.

The deuterated SDMA is added in a known amount. The ratio of the intensity of the mass spectrum peak having an m/z ratio of 172 derived from a parent ion having an m/z ratio 203 to the intensity of the mass spectrum peak having an m/z ratio of 175 derived from a parent ion having an m/z ratio 209, i.e., the ratio of the intensity 172/203 peak to the 175/209 peak, is used to determine the amount of SDMA in the sample.

For example if the intensity of the 172/203 peak is 100 and the intensity of the 175/210 peak is 200, the amount of SDMA in the sample is one-half the amount of deuterated SDMA that was added to the sample.

In one embodiment, the SDMA level is quantified by creating a standard curve prepared by adding a fixed concentration of deuterated SDMA (i.e., the internal standard) to a series of solutions containing SDMA of different concentrations, determining the intensity of the 172/203 peak relative to the intensity of the 175/209 peak for each solution, and plotting the intensity of the 172/203 peak relative to the intensity of the 175/209 peak vs. the concentration of SDMA. The sample is prepared so that it includes the same concentration of internal standard. The intensity of the 172/203 peak relative to the intensity of the 175/209 peak from the sample is determined and the concentration of SDMA in the sample is obtained from the standard curve.

The sample volume typically is less than about 50 μL, preferably less than about 25 μL, more preferably less than about 20 μL, most preferably less than about 15 μL. In one embodiment the sample volume is about 10 μL.

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the assay mixture is passed through an HPLC column to provide an eluant containing components of the assay mixture and at least a portion of the eluant is ionized in the mass spectrometer operated in the positive ion mode to detect the ion formed from the SDMA and the ion formed from the isotopically labelled SDMA.

In one embodiment, the assay mixture is passed through an HPLC column using HPLC conditions B.

An advantage of the method for assaying SDMA is that it can be performed simultaneously with the assay for alanine transaminase activity and/or aspartate transaminase activity. When the method involves simultaneously assaying for SDMA, alanine transaminase activity, and/or aspartate transaminase activity, the sample is adjusted to a pH of between about 6.0 and about 9.0, preferably between about 7.0 and about 8.0. In one embodiment, the pH is about 7.4. The incubation conditions and quenching step associated with the assay for alanine transaminase activity and aspartate transaminase activity do not interfere with the assay for SDMA.

As an example, the assay for SDMA is performed simultaneously with the assay for alanine transaminase activity (but using two separate modes of the mass spectrometer, i.e., positive ion mode and negative ion mode). In such a simultaneous assay the mass spectrometer could be configured to detect the intensity of the 172/203 peak (i.e., the daughter ion derived from SDMA), the intensity of the 175/209 peak (i.e., the daughter ion derived from the SDMA internal standard, i.e., deuterated SDMA), the intensity of the 43/88 peak (i.e., the daughter ion derived from the pyruvate formed by the action of alanine transaminase on the substrate (i.e., the ALT product), i.e., alanine labeled with ¹³C in the 1-position), and the intensity of the 44/89 peak (i.e., the daughter ion from derived from the pyruvate internal standard, i.e., pyruvate labeled with a ¹³C at the 1- and 2-positions).

Another advantage of the method for assaying SDMA is that it can be performed simultaneously with the assay for alkaline phosphatase activity. When the method involves simultaneously assaying for alkaline phosphatase activity and SDMA levels, the sample is adjusted to a pH of between about 9.0 and about 12.0, preferable between about 9.5 and about 11.0. In one embodiment, the pH is about 10.2. The incubation conditions and quenching step associated with the assay for alkaline phosphatase activity do not interfere with the assay for SDMA.

In the method for simultaneously assaying alkaline phosphatase activity and SDMA levels the mass spectrometer could be configured to detect the intensity of the 172/203 peak (i.e., the daughter ion derived from SDMA), the intensity of the 175/209 peak (i.e., the daughter ion derived from the SDMA internal standard, i.e., deuterated SDMA), the intensity of the 46/138 peak (i.e., the daughter ion from derived from p-nitrophenol formed by the action of alkaline phosphatase on the substrate, i.e., the ALP product), and the intensity of the 44/144 peak (i.e., the daughter ion from derived from the internal standard, i.e., p-nitrophenol labeled with ¹³C at each of the carbon atoms).

### Method for Simultaneously Assaying for ALT Activity, AST Activity, ALP Activity, Glucose Levels, Urea Levels, Creatinine Levels, ADMA Levels and SDMA Levels

The above assays can be combined to provide a method for simultaneously assaying for ALT activity, AST activity, ALP activity, glucose levels, urea levels, creatinine levels, ADMA levels, and SDMA levels.

The method involves:
(i) providing a sample suspected of containing one or more of: alanine transaminase, aspartate transaminase, alkaline phosphatase, glucose, urea, creatinine, ADMA, and SDMA;
(ii) dividing the sample into a first portion and a second portion;
(iii) contacting the first portion with (a) a substrate for alanine transaminase, wherein the substrate for alanine transaminase comprises a first isotopic label and (b) a substrate for aspartate transaminase, wherein the substrate for aspartate transaminase comprises a second isotopic label;
(iv) contacting the second portion with a substrate for alkaline phosphatase;
(v) adding isotopically labelled glucose comprising a third isotopic label; isotopically labelled urea comprising a fourth isotopic label, isotopically labelled creatinine comprising a fifth isotopic label, isotopically labelled ADMA comprising a sixth isotopic label, and isotopically labelled SDMA comprising a seventh isotopic label to at least one of the first portion or the second portion;
(vi) combining the first portion and the second portion to provide an assay mixture;
(vii) passing a first quantity of the assay mixture through an HPLC column to provide a first eluant containing components of the assay mixture;
(viii) analyzing a portion of the first eluant with a mass spectrometer, wherein the mass spectrometer is operated in the negative ion mode, to:
   (a) generate and detect an ion formed from a metabolite resulting from the action of the alanine transaminase on the substrate for alanine transaminase, wherein the metabolite resulting from the action of the alanine transaminase on the substrate for alanine transaminase comprises the first isotopic label,
   (b) generate and detect an ion formed from a metabolite resulting from the action of the aspartate transaminase on the substrate for aspartate transaminase, wherein the metabolite resulting from the action of the aspartate transaminase on the substrate for aspartate transaminase comprises the second isotopic label,
   (c) generate and detect an ion formed from a metabolite resulting from the action of the alkaline phosphatase on the substrate for alkaline phosphatase,
   (d) generate and detect an ion formed from the glucose,
   (e) generate and detect an ion formed from the isotopically labelled glucose, wherein the ion formed from the isotopically labelled glucose comprises the third isotopic label;
(ix) passing a second quantity of the assay mixture through an HPLC column to provide a second eluant containing the components of the assay mixture;
(x) analyzing a portion of the second eluant with a mass spectrometer, wherein the mass spectrometer is operated in the positive ion mode, to:
   (a) generate and detect an ion formed from the urea,
   (b) generate and detect an ion formed from the isotopically labelled urea, wherein the ion formed from the isotopically labelled urea comprises the fourth isotopic label,
   (c) generate and detect an ion formed from the creatinine,
   (d) generate and detect an ion formed from the isotopically labelled creatinine, wherein the ion formed from the isotopically labelled creatinine comprises the fifth isotopic label,
   (e) generate and detect an ion formed from the ADMA,
   (f) generate and detect an ion formed from the isotopically labelled ADMA, wherein the ion formed from the isotopically labelled ADMA comprises the sixth isotopic label,
   (g) generate and detect an ion formed from the SDMA, and
   (h) generate and detect an ion formed from the isotopically labelled SDMA, wherein the ion formed from the isotopically labelled SDMA comprises the seventh isotopic label.

In one embodiment, the sample volume is less than about 50 μL, preferably less than about 40 μL, more preferably less than about 30 μL. In one embodiment, the sample volume is about 25 μL.

In one embodiment, the volume of the first portion and the second portion is each less than about 20 μL, more preferably less than about 15 μL. In one embodiment, the volume of the first portion and the second portion is each about 10 μL.

The small volume required for the assay is advantageous. For example, if ALT activity, AST activity, ALP activity, glucose levels, urea levels, creatinine levels, ADMA levels and SDMA levels are being measured in the blood of a laboratory animal, such as a mouse, the small volume allows a determination to be made without sacrificing the animal.

In one embodiment, the substrate for alanine transaminase is alanine labeled with ¹³C in the 1-position, i.e.,

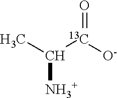

and the 43/88 peak is used to detect the ion formed from the metabolite resulting from the action of the alanine transaminase on the substrate for alanine transaminase, as discussed above in the section entitled “Assay for alanine transaminase activity.”

In one embodiment, the first portion further includes pyruvate labeled with ¹³C at the 1- and 2-positions, i.e.,

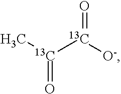

as an internal standard, and the ratio of the intensity of the 43/88 peak to the intensity of the 44/89 peak is used to determine the amount of pyruvate formed by the alanine transaminase catalyzed conversion of alanine to pyruvate, as discussed above in the section entitled “Assay for alanine transaminase activity.”

In one embodiment, the substrate for aspartate transaminase is aspartate labeled with ¹³C in the 1-, 2-, 3-, and 4-position and with ¹⁵N at the nitrogen, i.e.,

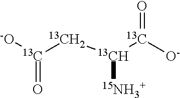

and the 45/90 peak is used to detect the ion formed from the metabolite resulting from the action of the aspartate transaminase on the substrate for aspartate transaminase, as discussed above in the section entitled “Assay for aspartate transaminase activity.”

In one embodiment, the first portion further includes pyruvate labeled with ¹³C at the 1- and 2-positions, i.e.,

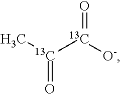

as an internal standard, and the ratio of the intensity of the 45/90 peak to the intensity of the 44/89 peak is used to determine the amount of pyruvate formed by the aspartate transaminase catalyzed conversion of aspartate to pyruvate, as discussed above in the section entitled “Assay for aspartate transaminase activity.”

In one embodiment, the pH of the first portion is adjusted to a pH of between about 6.0 and about 9.0, preferable between about 6.5 and about 8.5. In one embodiment, the pH is about 7.4.

In one embodiment, α-ketoglutarate and pyridoxal phosphate (PLP), which are cofactors for the reaction catalyzed by alanine transaminase and aspartate transaminase, are added to the first portion.

In one embodiment, the first portion further includes oxaloacetate decarboxylase to assure that oxaloacetate, which forms from the action of the aspartate transaminase on the substrate for aspartate transaminase, undergoes complete decarboxylation to provide isotopically labelled pyruvate.

As discussed above in the section entitled “Assay for alanine transaminase activity” and the section entitled “Assay for aspartate transaminase activity,” in one embodiment, the first portion is incubated for a given amount of time, typically at a constant temperature, so that the enzymatic conversion of the substrate(s) to the metabolite can take place. After the allotted time has elapsed, the assay mixture is quenched to stop the enzymatic reaction.

In one embodiment, the substrate for alkaline phosphatase is p-nitrophenyl phosphate, e.g.,

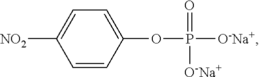

and the 46/138 peak is used to detect the ion formed from the metabolite resulting from the action of the alkaline phosphatase on the substrate for alkaline phosphatase, as discussed above in the section entitled “Assay for alkaline phosphatase activity.”

In one embodiment, magnesium and zinc are combined with the substrate for alkaline phosphatase.

In one embodiment, the second portion further includes p-nitrophenol labeled with ¹³C at each of the carbon atoms, i.e.,

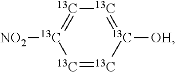

as an internal standard and the ratio of the intensity of the 46/138 peak to the intensity of the 46/144 peak is used to determine the amount of p-nitrophenol formed by the alkaline phosphatase catalyzed conversion of p-nitrophenyl phosphate, as discussed above in the section entitled “Assay for alkaline phosphatase activity.”

In one embodiment, the pH of the first portion is adjusted to a pH of between about 9.0 and about 12.0, preferable between about 9.5 and about 11.0. In one embodiment, the pH is about 10.2.

As discussed above in the section entitled “Assay for alkaline phosphatase activity,” in one embodiment, the second portion is incubated for a given amount of time, typically at a constant temperature, so that the enzymatic conversion of the substrate to the metabolite can take place. After the allotted time has elapsed, the assay mixture is quenched to stop the enzymatic reaction.

In one embodiment, the isotopically labelled glucose is ¹³C-labeled glucose, i.e.,

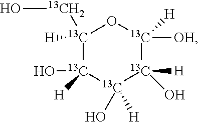 [SMILES: O[13CH2][13C@H]1O[13C@@H](O)[13C@H](O)[13C@@H](O)[13C@@H]1O]

the 119/179 peak is used to detect the ion formed from glucose, the 123/185 peak is used to detect the ion formed from the isotopically labelled glucose (i.e., ¹³C-labeled glucose), and the ratio of the intensity of the 119/179 peak to the intensity of the 123/185 peak is used to determine the amount of glucose in the sample, as discussed above in the section entitled “Assay for glucose.”

In one embodiment, the isotopically labelled urea is urea labeled with ¹³C and ¹⁵N, i.e.,

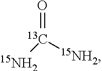

the 44/61 peak is used to detect the ion formed from urea, the 46/64 peak is used to detect the ion formed from the isotopically labelled urea (i.e., urea labeled with ¹³C and ¹⁵N), and the ratio of the intensity of the 44/61 peak to the intensity of the 46/64 peak is used to determine the amount of urea in the sample, as discussed above in the section entitled “Assay for urea.”

In one embodiment, the isotopically labelled creatinine is creatinine with a deuterated methyl group, i.e.,

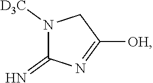 [SMILES: [2H]C([2H])([2H])N1CC(O)=NC1=N]

the 44/114 peak is used to detect the ion formed from creatinine, the 47/117 peak is used to detect the ion formed from the isotopically labelled creatinine (i.e., creatinine with a deuterated methyl group), and the ratio of the intensity 44/114 peak to the 47/117 peak is used to determine the amount of creatinine in the sample, as discussed above in the section entitled “Assay for creatinine.”

In one embodiment, the isotopically labelled creatinine is creatinine with a deuterated methyl group, the 86/114 peak is used to detect the ion formed from creatinine, the 89/117 peak is used to detect the ion formed from the isotopically labelled creatinine (i.e., creatinine with a deuterated methyl group), and the ratio of the intensity 86/114 peak to the 89/117 peak is used to determine the amount of creatinine in the sample, as discussed above in the section entitled “Assay for creatinine.”

In one embodiment, the isotopically labelled ADMA is deuterated ADMA, i.e.:

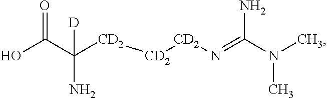

the 46/203 peak is used to detect the ion formed from ADMA, the 46/210 peak is used to detect the ion formed from the isotopically labelled ADMA (i.e., deuterated ADMA), and the ratio of the intensity 46/203 peak to the 46/210 peak, is used to determine the amount of ADMA in the sample, as discussed above in the section entitled “Assay for ADMA.”

In one embodiment, the isotopically labelled SDMA is deuterated SDMA, i.e.:

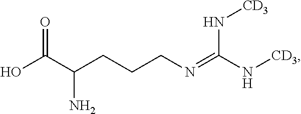 [SMILES: [2H]C([2H])([2H])NC(=NCCCC(N)C(=O)O)NC([2H])([2H])[2H]]

the 172/203 peak is used to detect the ion formed from the SDMA, the 175/209 peak is used to detect the ion formed from the isotopically labelled SDMA (i.e., deuterated SDMA), and the ratio of the intensity 172/203 peak to the 175/209 peak, is used to determine the amount of SDMA in the sample, as discussed above in the section entitled “Assay for SDMA.”

In one embodiment, the method involves:
(i) providing a sample suspected of containing one or more of: alanine transaminase, aspartate transaminase, alkaline phosphatase, glucose, urea, creatinine, ADMA, and SDMA;
(ii) dividing the sample into a first and a second portion;
(iii) contacting the first portion with:
   (a) α-ketoglutarate,
   (b) pyridoxal phosphate,

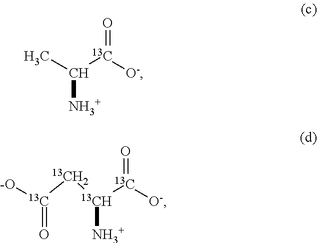

and
(e) oxaloacetate decarboxylase,
   to provide a first reaction mixture;
(iv) contacting the second portion with:

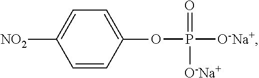

and
(v) optionally, zinc sulfate, magnesium acetate, and HEDTA,
   to provide a second reaction mixture;
(vi) adding:

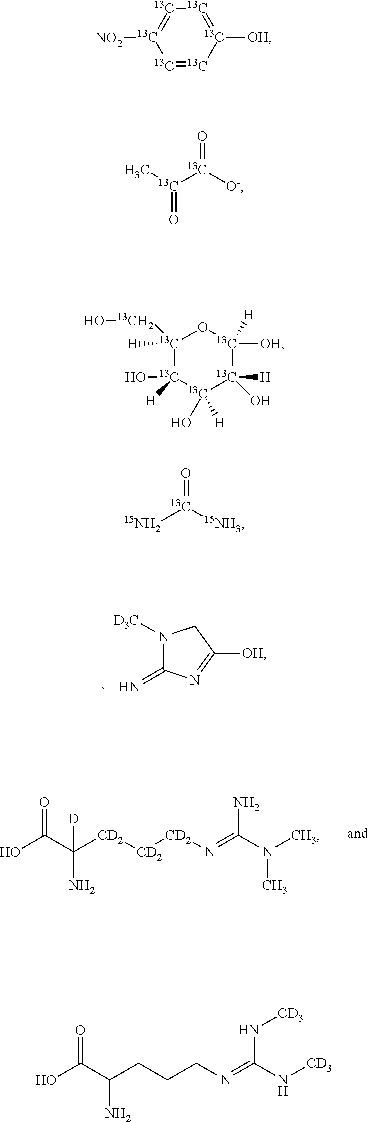

to at least one of the first reaction mixture or the second reaction mixture;
(vii) allowing the first reaction mixture to incubate at a temperature for a period of time;
(viii) quenching the first reaction mixture to provide a quenched first reaction mixture;
(ix) allowing the second reaction mixture to incubate at a temperature for a period of time;
(x) quenching the second reaction mixture to provide a quenched second reaction mixture;
(xi) combining the quenched first reaction mixture and the quenched second reaction mixture to provide an assay mixture;
(xii) passing a first quantity of the assay mixture through an HPLC column to provide a first eluant containing components of the assay mixture;
(xiii) analyzing at least a portion of the first eluant with a mass spectrometer, wherein the mass spectrometer is operated in the negative ion mode, to provide a first plurality of parent ions;
(xiv) separating parent ions having an m/z ration of 88, 90, 138, 179, and 185 from the first plurality of parent ions;
(xv) fragmenting the parent ions having an m/z ratio of 88, 90, 138, 179, and 185 to provide a first plurality of daughter ions;
(xvi) separating daughter ions having an m/z ratio of 43, 45, 46, 119, and 123 from the first plurality of daughter ions;
(xvii) detecting the intensity of the daughter ions that have an m/z ratio of 43, 45, 46, 119, and 123;
(xviii) passing a second quantity of the assay mixture through an HPLC column to provide a second eluant containing components of the assay mixture;
(xix) analyzing at least a portion of the second eluant with a mass spectrometer, wherein the mass spectrometer is operated in the positive ion mode, to provide a second plurality of parent ions;
(xx) separating parent ions having an m/z ration of 61, 64, 114, 117, 203, 209, and 210 from the second plurality of parent ions;
(xxi) fragmenting the parent ions having an m/z ratio of 61, 64, 114, 117, 203, 209, and 210 to provide a second plurality of daughter ions;
(xxii) separating daughter ions having an m/z ratio of 44, 46, 47, 86, 89, 172, and 175 from the second plurality of daughter ions; and
(xxiii) detecting the intensity of the daughter ions that have an m/z ratio of 44, 46, 47, 86, 89, 172, and 175.

The 43/88 peak is indicative of alanine transaminase activity, which can be quantified as described above in the section entitled “Assay for alanine transaminase activity.”

The 45/90 peak is indicative of aspartate transaminase activity, which can be quantified as described above in the section entitled “Assay for aspartate transaminase activity.”

The 46/138 peak is indicative of alkaline phosphatase activity, which can be quantified as described above in the section entitled “Assay for alkaline phosphatase activity.”

The ratio of the intensity of the 119/179 peak to the intensity of the 123/185 peak is used to determine the amount of glucose in the sample, as described above in the section entitled “Assay for glucose.”

The ratio of the intensity of the 44/61 peak to the intensity of the 46/64 peak is used to determine the amount of urea in the sample, as described above in the section entitled “Assay for urea.”

The ratio of the intensity 44/114 peak to the 47/117 peak or the ratio of the intensity 86/114 peak to the 89/117 peak, is used to determine the amount of creatinine in the sample, as described above in the section entitled “Assay for creatinine.”

The ratio of the intensity 46/203 peak to the 46/210 peak, is used to determine the amount of ADMA in the sample, as described above in the section entitled “Assay for ADMA.”

The ratio of the intensity 172/203 peak to the 175/209 peak, is used to determine the amount of SDMA in the sample, as described above in the section entitled “Assay for SDMA.”

Suitable samples include, but are not limited to, blood, serum, plasma, urine, tissue (such as liver or kidney) homogenates, feces, sweat, saliva, spinal fluid, and synovial fluid. In one embodiment, the sample is serum.

In one embodiment, the first quantity of the assay mixture is passed through the HPLC column using HPLC conditions A and the second quantity of the assay mixture is passed through the HPLC column using HPLC conditions B.

The method is specific, accurate, and sensitive and uses only a small volume of sample.

The method allows the concentration of each analyte to be measured over a broad range. For example, AST activity can be measured over a range of from about 7 to about 4120 units/L, ALT activity can be measured over a range of from about 12 to about 7373 units/L, ALP activity can be measured over a range of from about 7 to about 4448 units/L, glucose levels can be measured over a range of about 2.5 to about 1500 mg/dL, creatinine levels can be measured over a range of about 0.01 to about 6.0 mg/dL, urea levels can be measured over a range of about 5 to about 3000 mg/dL, ADMA levels can be measured over a range of about 0.25 to about 150 ug/dL, and SDMA levels can be measured over a range of about 0.25 to about 150 ug/dL.

The method is also rapid. For example, when the first quantity of the assay mixture is passed through the HPLC column using HPLC conditions A the total run time is about 2.5 minutes. Similarly, when the second quantity of the assay mixture is passed through the HPLC column using HPLC conditions B the total run time is about 2.5 minutes. Thus, a sample can be analyzed in less than about 6 minutes (i.e., 2 injections with less than a 3 minute run time). The short analysis time of the method allows a large number of samples to be run in a short period of time. For example, using the method allows as many as about 120 samples per day to be analyzed.

A person of ordinary skill in the art would understand that rather than using unit values for m/z ratios of the parent ions and fragment ions, the assays could be performed using more accurate m/z values for these ions, e.g., an accuracy to the tenth decimal place.

### EXAMPLES

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1: Analysis of Serum Samples to Determine ALT, AST, and ALP Enzyme Activity and to Quantify Glucose, Urea, Creatinine, SDMA, and ADMA Levels

In order to quantify the activity of each enzyme and the levels of glucose, urea, creatinine, SDMA, and ADMA (i.e., the small molecules) from a serum sample, standard curves were used. The standards curves were generated from calibration standards.

The analysis quantifies the activity of three enzymes (ALT, AST, and ALP) in a given serum sample. AST and ALT enzymes exhibit optimum enzyme activity at a pH of about 7.4 and ALP exhibits optimum activity at a pH of about 10.2. Therefore, the calibration standards for ALT and AST and the assay for AST and ALT enzyme activity is conducted at a pH of about 7.4 and the calibration standards for ALP and the assay for ALP activity is conducted at a pH of about 10.4.

### Preparation of Calibration Standards:

The following is a list of the materials and equipment necessary to complete the preparation of the calibration standards.
Stripped/dialyzed serum of desired species
Methanol, Optima™ LC/MS Grade (commercially available from Thermo Fisher Scientific of Waltham, MA), or similar.
Deionized Water
Creatinine anhydrous, ≥98% (commercially available from Sigma Aldrich of St. Louis, MO) or similar
Urea, ReagentPlus®, ≥99.5%, pellets (commercially available from Sigma Aldrich of St. Louis, MO), or similar
SDMA (commercially available from Sigma Aldrich of St. Louis, MO), or similar
ADMA (commercially available from Sigma Aldrich of St. Louis, MO), or similar
Glucose (commercially available from Sigma Aldrich of St. Louis, MO), or similar
4-Nitrophenol, ReagentPlus®, ≥99% (commercially available from Sigma Aldrich of St. Louis, MO), or similar (i.e., the ALP product)
Sodium pyruvate (1-¹³C, 99%); (commercially available from Cambridge Isotope Laboratories, Inc. of Tewksbury, MA), or similar (i.e., the ALT product)
Sodium pyruvate (¹³C3, 99%) (commercially available from Cambridge Isotope Laboratories, Inc. of Tewksbury, MA) or similar (i.e., the AST product)
Alanine Aminotransferase, Porcine Heart (commercially available from LeeBio Solutions of Maryland Heights, MO), or similar
Aspartate Aminotransferase, Porcine Heart (commercially available from LeeBio Solutions of Maryland Heights, MO), or similar
Alkaline Phosphatase from bovine intestinal mucosa, commercially available from Sigma Aldrich of St. Louis, MO), or similar
Analytical balance capable of measuring 0.01 mg
Suitable pipettes
Suitable small volume tube ware
Laboratory glassware, Class A

### Preparation of Individual Stock Solutions of Each Analyte:

Weigh each of the following into 5.0 mL capped glass vials:
Glucose stock solution: Weigh out approximately 1000 mg powder and dissolve in deionized water to a concentration of 1000 mg/mL. Run under hot water to lightly heat to completely dissolve, be sure vial is tightly capped;
ALT product stock solution: Weigh out approximately 50-60 mg powder and dissolve in deionized water to provide a concentration of about 500 mg/mL;
AST product stock solution: Weigh out approximately 50-60 mg powder and dissolve in deionized water to provide a concentration of about 500 mg/mL;
4-Nitrophenol stock solution: Weigh out approximately 200-300 mg powder and dilute in methanol to provide a concentration of about 300 mg/mL;
Urea stock solution: Weigh out approximately 1000 mg of powder and, dissolve in deionized water to provide a concentration of about 1000 mg/mL;
Creatinine stock solution: Weigh out approximately 10-20 mg powder and dissolve in deionized water to provide a concentration of about 8 mg/mL;
SDMA/ADMA stock solution: Weigh out approximately 10-20 mg of SDMA and ADMA into separate vials and dissolve in deionized water to provide a concentration of about 8 mg/mL. Next, prepare a single 800 μg/mL solution from these 80 mg/mL solutions by mixing 1.0 mL of each solution with 8 mL of deionized water. Rather than prepare an SDMA/ADMA stock solution, one can prepare separate SDMA and ADMA stock solutions.

### Preparation of Intermediate Solutions

Next, two intermediate solutions are prepared by adding each individual stock solution to a 1.5 mL microcentrifuge tube as follows.

Intermediate solution (Working CS 12-pH 7.4) used for analysis at pH 7.4 is prepared as follows:
Glucose stock solution: 200 μL
ALT product stock solution: 175 μL
AST product stock solution: 100 μL
Urea stock solution: 400 μL
Creatinine stock solution: 100 μL
SDMA and ADMA stock solution: 25 μL
Vortex vigorously

Intermediate solution (Working CS 12-pH 10.2) used for analysis at pH 10.2 is prepared as follows:
Glucose stock solution: 200 μL
4-Nitrophenol stock solution 1:275 μL
Urea stock solution: 400 μL
Creatinine stock solution: 100 μL
SDMA and ADMA stock solution: 25 μL
Vortex vigorously

### Preparation of Working Calibration Solutions and Calibration Standards

Next, two sets of working calibration solutions (“CS”) were prepared from each of the intermediate solutions. One set of working CSs is for analysis at pH 7.4 (i.e., working CS #-pH 7.4) and one set of working CSs is for analysis at pH 10.2 (i.e., working CS #-pH 10.2). The working CSs were prepared as follows.

The set of working CSs used for analysis at pH 7.4 were prepared as follows:
a. Working CS 11-pH7.4:100 μL of working CS 12-pH 7.4+100 μL DI water
b. Working CS 10-pH7.4:100 μL of working CS 12-pH 7.4+220 μL DI water
c. Working CS 9-pH7.4:100 μL of working CS 12-pH 7.4+450 μL DI water
d. Working CS 8-pH7.4:50 μL of working CS 12-pH 7.4+350 μL DI water
e. Working CS 7-pH7.4:50 μL of working CS 12-pH 7.4+450 μL DI water
f. Working CS 6-pH7.4:20 μL of working CS 12-pH 7.4+300 μL DI water
g. Working CS 5-pH7.4:20 μL of working CS 12-pH 7.4+380 μL DI water
h. Working CS 4-pH7.4:20 μL of working CS 12-pH 7.4+620 μL DI water
i. Working CS 3-pH7.4:15 μL of working CS 12-pH 7.4+980 μL DI water
j. Working CS 2-pH7.4:10 μL of working CS 12-pH 7.4+1990 μL DI water
k. Working CS 1-pH7.4:5 μL of working CS 12-pH 7.4+2000 μL DI water

| | ALT | AST | | | | | |
|---|---|---|---|---|---|---|---|
| | product | product | Glucose | Creatinine | Urea | SDMA | ADMA |
| Working CS | (μg/mL) | (μg/mL) | (μg/mL) | (μg/mL) | (μg/mL) | (μg/mL) | (μg/mL) |
| Working CS 1-pH7.4 | 218 | 125 | 499 | 2.00 | 998 | 0.05 | 0.05 |
| Working CS 2-pH7.4 | 438 | 250 | 1000 | 4.00 | 2000 | 0.10 | 0.10 |
| Working CS 3-pH7.4 | 1319 | 754 | 3015 | 12.06 | 6030 | 0.30 | 0.30 |
| Working CS 4-pH7.4 | 2734 | 1563 | 6250 | 25.00 | 12500 | 0.63 | 0.63 |
| Working CS 5-pH7.4 | 4375 | 2500 | 10000 | 40.00 | 20000 | 1.00 | 1.00 |
| Working CS 6-pH7.4 | 5469 | 3125 | 12500 | 50.00 | 25000 | 1.25 | 1.25 |
| Working CS 7-pH7.4 | 8750 | 5000 | 20000 | 80.00 | 40000 | 2.00 | 2.00 |
| Working CS 8-pH7.4 | 10938 | 6250 | 25000 | 100.00 | 50000 | 2.50 | 2.50 |
| Working CS 9-pH7.4 | 15909 | 9091 | 36364 | 145.45 | 72727 | 3.64 | 3.64 |
| Working CS 10-pH7.4 | 27344 | 15625 | 62500 | 250.00 | 125000 | 6.25 | 6.25 |
| Working CS 11-pH7.4 | 43750 | 25000 | 100000 | 400.00 | 200000 | 10.00 | 10.00 |
| Working CS 12-pH7.4 | 87500 | 50000 | 200000 | 800.00 | 400000 | 20.00 | 20.00 |

The final concentration of each analyte in each working CS is as follows.

Next, calibration standards used for the analysis at pH 7.4 were prepared in stripped mouse serum in a series of 1.5 mL microcentrifuge tubes using the working CSs prepared above. The following procedure was used to prepare the calibration standards:
1. Add 185 μL stripped serum to tubes designated as Serum CS11 and 12.
2. Add 190 μL stripped serum to tubes designated as Serum CS 1-10.
3. Individually add 15 μL of Working CS 11 and Working CS12 (each containing 185 μL of serum) to Serum CS 11 and Serum CS 12, respectively.
4. Individually add 10 μL of each Working CS 1-10 to Serum CS 1-10 (containing 190 u L of serum), respectively.
5. Vortex after each addition.
6. Volumes can be adjusted as needed.
7. If not for immediate use, aliquot and freeze at −80° C.

The final concentration of each analyte in the series of calibration standards is as follows:

| | ALT | AST | | | | | |
|---|---|---|---|---|---|---|---|
| | product | product | Glucose | Creatinine | Urea | SDMA | ADMA |
| Serum CS | (μg/mL) | (μg/mL) | (mg/dL) | (mg/dL) | (mg/dL) | (μg/mL) | (μg/mL) |
| Serum CS 1-pH7.4 | 10.91 | 6.23 | 2.49 | 0.01 | 4.99 | 0.25 | 0.25 |
| Serum CS 2-pH7.4 | 21.88 | 12.50 | 5.00 | 0.02 | 10.00 | 0.50 | 0.50 |
| Serum CS 3-pH7.4 | 65.95 | 37.69 | 15.08 | 0.06 | 30.15 | 1.51 | 1.51 |
| Serum CS 4-pH7.4 | 136.72 | 78.13 | 31.25 | 0.13 | 62.50 | 3.13 | 3.13 |
| Serum CS 5-pH7.4 | 218.75 | 125.00 | 50.00 | 0.20 | 100.00 | 5.00 | 5.00 |
| Serum CS 6-pH7.4 | 273.44 | 156.25 | 62.50 | 0.25 | 125.00 | 6.25 | 6.25 |
| Serum CS 7-pH7.4 | 437.50 | 250.00 | 100.00 | 0.40 | 200.00 | 10.00 | 10.00 |
| Serum CS 8-pH7.4 | 546.88 | 312.50 | 125.00 | 0.50 | 250.00 | 12.50 | 12.50 |
| Serum CS 9-pH7.4 | 795.45 | 454.55 | 181.82 | 0.73 | 363.64 | 18.18 | 18.18 |
| Serum CS 10-pH7.4 | 1367.19 | 781.25 | 312.50 | 1.25 | 625.00 | 31.25 | 31.25 |
| Serum CS 11-pH7.4 | 3281.25 | 1875.00 | 750.00 | 3.00 | 1500.00 | 75.00 | 75.00 |
| Serum CS 12-pH7.4 | 6562.50 | 3750.00 | 1500.00 | 6.00 | 3000.00 | 150.00 | 150.00 |

A set of working CSs used for analysis at pH 10.2 were prepared as follows:
a. Working CS 11-pH 10.2:100 μL of working CS 12-pH 10.2+100 μL DI water
b. Working CS 10-pH 10.2:100 μL of working CS 12-pH 10.2+220 μL DI water
c. Working CS 9-pH 10.2:100 μL of working CS 12-pH 10.2+450 μL DI water
d. Working CS 8-pH 10.2:50 μL of working CS 12-pH 10.2+350 μL DI water
e. Working CS 7-pH 10.2:50 μL of working CS 12-pH 10.2+450 μL DI water
f. Working CS 6-pH 10.2:20 μL of working CS 12-pH 10.2+300 μL DI water
g. Working CS 5-pH 10.2:20 μL of working CS 12-pH 10.2+380 μL DI water
h. Working CS 4-pH 10.2:20 μL of working CS 12-pH 10.2+620 μL DI water
i. Working CS 3-pH 10.2:15 μL of working CS 12-pH 10.2+980 μL DI water
j. Working CS 2-pH 10.2:10 μL of working CS 12-pH 10.2+1990 μL DI water
k. Working CS 1-pH 10.2:5 μL of working CS 12-pH 10.2+2000 μL DI water

The final concentration of each analyte in each working CS is as follows:

| | 4-Nitro | | | | | |
|---|---|---|---|---|---|---|
| | phenol | Glucose | Creatinine | Urea | SDMA | ADMA |
| Working CS | (μg/mL) | (μg/mL) | (μg/mL) | (μg/mL) | (μg/mL) | (μg/mL) |
| Working CS 1-pH10.2 | 206 | 499 | 2.00 | 998 | 0.05 | 0.05 |
| Working CS 2-pH10.2 | 413 | 1000 | 4.00 | 2000 | 0.10 | 0.10 |
| Working CS 3-pH10.2 | 1244 | 3015 | 12.06 | 6030 | 0.30 | 0.30 |
| Working CS 4-pH10.2 | 2578 | 6250 | 25.00 | 12500 | 0.63 | 0.63 |
| Working CS 5-pH10.2 | 4125 | 10000 | 40.00 | 20000 | 1.00 | 1.00 |
| Working CS 6-pH10.2 | 5156 | 12500 | 50.00 | 25000 | 1.25 | 1.25 |
| Working CS 7-pH10.2 | 8250 | 20000 | 80.00 | 40000 | 2.00 | 2.00 |
| Working CS 8-pH10.2 | 10313 | 25000 | 100.00 | 50000 | 2.50 | 2.50 |
| Working CS 9-pH10.2 | 15000 | 36364 | 145.45 | 72727 | 3.64 | 3.64 |
| Working CS 10-pH10.2 | 25781 | 62500 | 250.00 | 125000 | 6.25 | 6.25 |
| Working CS 11-pH10.2 | 41250 | 100000 | 400.00 | 200000 | 10.00 | 10.00 |
| Working CS 12-pH10.2 | 82500 | 200000 | 800.00 | 400000 | 20.00 | 20.00 |

Next, calibration standards used for the analysis at pH 10.2 were prepared in stripped mouse serum in a series of 1.5 mL microcentrifuge tubes using the working CSs prepared above. The following procedure was used to prepare the calibration standards:
1. Add 185 μL stripped serum to tubes designated Serum CS 11 and 12.
2. Add 190 μL stripped serum to tubes designated as Serum CS 1-10.
3. Individually add 15 μL of Working CS 11 and Working CS12 (each containing 185 μL of serum) to Serum CS 11 and Serum CS 12, respectively.
4. Individually add 10 μL of each Working CS 1-10 to Serum CS 1-10 (containing 190 μL of serum), respectively.
5. Vortex after each addition.
6. Volumes can be adjusted as needed.
7. If not for immediate use, aliquot and freeze at −80° C.

The final concentration of each analyte in the series of calibration standards is as follows:

| | 4-nitro | | | | | |
|---|---|---|---|---|---|---|
| | phenol | Glucose | Creatinine | Urea | SDMA | ADMA |
| Serum CS | (μg/mL) | (mg/dL) | (mg/dL) | (mg/dL) | (μg/mL) | (μg/mL) |
| Serum CS 1-pH10.2 | 10.29 | 2.49 | 0.01 | 4.99 | 0.25 | 0.25 |
| Serum CS 2-pH10.2 | 20.63 | 5.00 | 0.02 | 10.00 | 0.50 | 0.50 |
| Serum CS 3-pH10.2 | 62.19 | 15.08 | 0.06 | 30.15 | 1.51 | 1.51 |
| Serum CS 4-pH10.2 | 128.91 | 31.25 | 0.13 | 62.50 | 3.13 | 3.13 |
| Serum CS 5-pH10.2 | 206.25 | 50.00 | 0.20 | 100.00 | 5.00 | 5.00 |
| Serum CS 6-pH10.2 | 257.81 | 62.50 | 0.25 | 125.00 | 6.25 | 6.25 |
| Serum CS 7-pH10.2 | 412.50 | 100.00 | 0.40 | 200.00 | 10.00 | 10.00 |
| Serum CS 8-pH10.2 | 515.63 | 125.00 | 0.50 | 250.00 | 12.50 | 12.50 |
| Serum CS 9-pH10.2 | 750.00 | 181.82 | 0.73 | 363.64 | 18.18 | 18.18 |
| Serum CS 10-pH10.2 | 1289.06 | 312.50 | 1.25 | 625.00 | 31.25 | 31.25 |
| Serum CS 11-pH10.2 | 3093.75 | 750.00 | 3.00 | 1500.00 | 75.00 | 75.00 |
| Serum CS 12-pH10.2 | 6187.50 | 1500.00 | 6.00 | 3000.00 | 150.00 | 150.00 |

### Internal Standard Preparation:

The internal standard solution was prepared by dissolving powdered forms (or a stock solutions purchased from a vendor) of isotopically labeled urea, creatinine, SDMA, ADMA, glucose, 4-nitrophenol, and pyruvate in deionized water.

The following isotopically labelled compounds were used:
Urea labelled with ¹³C and ¹⁵N (¹³C, 99%; ¹⁵N2, 98%+) (commercially available from Cambridge Isotope Laboratories, Inc. of Tewksbury, MA, catalog CNLM-234-PK)
Sodium pyruvate-1,2-¹³C2 (commercially available from Sigma Aldrich of St. Louis, MO, catalog number 493392-500 MG)
4-Nitrophenol-¹³C6 (commercially available from Sigma Aldrich of St. Louis, MO, catalog number 768499)
Deuterated ADMA (d7-ADMA), >98% (commercially available from Cambridge Isotope Laboratories, Inc. of Tewksbury, MA, catalog number DLM-7476-PK)
Deuterated SDMA (commercially available from Toronto Research Chemicals of North York, ON, Canada, catalog number D463582),
Creatinine with a deuterated methyl group (creatinine D3, 98%) (commercially available from Cambridge Isotope Laboratories, Inc. of Tewksbury, MA, catalog number DLM-3653-PK)
¹³C labelled glucose (D-GLUCOSE (U-¹³C6, 99%) (commercially available from Cambridge Isotope Laboratories, Inc. of Tewksbury, MA, catalog number CLM-1396-PK)

The final concentrations of each isotopically labeled compound in the internal standard solution is approximately as follows:
SDMA—0.5 μg/mL,
ADMA—0.75 μg/mL,
Urea—220 μg/mL,
Creatinine—1.0 μg/mL,
Pyruvate—85 μg/mL,
Glucose—1000 μg/mL,
4-nitrophenol—30 μg/mL.

### Standard Curves for Measuring AST, ALT, and ALP Activity

### ALT and AST Activity:

For measuring AST and ALT activity a buffer was used to maintain a pH of about 7.4. 10.0 μL of calibration standards Serum CS 1-pH7.4 to Serum CS 12-pH7.4 were pipetted into designated wells (i.e., wells 1 to 12) of a first 96 well extraction plate (commercially available from VWR International of Radnor, PA). Next, 300 μL of methanol was immediately added to each well. Then each of the following solutions were added to each of the wells:
1. 10.0 μL of oxaloacetic acid decarboxylase enzyme dissolved in 100 mM Tris-10 mM ammonium bicarbonate in water pH about 7.4. The solution of oxaloacetic acid decarboxylase is prepared to have a final activity level of 1000 U/L based on the certificate of analysis provided by the vendor. This is accomplished by adjusting the weight of the enzyme
2. 15.0 μL of alanine labelled with ¹³C in the 1-position dissolved in 100 mM Tris-10 mM ammonium bicarbonate in water at a pH of about 7.4. The concentration of alanine labelled with ¹³C in the 1-position is 55 mg/mL (prepared by dissolving 100 mg of alanine labelled with ¹³C in the 1-position in 1.8 mL of 100 mM Tris-10 mM Ammonium Bicarbonate in water at pH 7).
3. 65.0 μL of a solution containing pyridoxal 5′-phosphate monohydrate (0.1 mg/mL), α-ketoglutaric acid (8.8 mg/mL), and L-aspartic acid-¹³C4,15N (3.1 mg/mL) in 100 mM Tris-10 mM ammonium bicarbonate in water pH about 7.4.
4. 20.0 μL of internal standard.

After the four solutions were added to each well, the resulting mixture was agitated by pipetting up and down 20-30× so as to precipitate proteins followed by centrifuging for about 10 minutes at a relative centrifugal force (RCF) of about 5000 to provide a pellet at the bottom of each well.

### ALP Activity:

For measuring ALP enzyme activity, a buffer is used to maintain a pH of about 10.2. 10.0 μL of calibration standards Serum CS 1-pH10.2 to Serum CS 12-pH10.2 were pipetted into designated wells (i.e., wells 1 to 12) of a second 96 well extraction plate. Next, 300 μL of methanol was immediately added to each well followed by 90.0 μL of para-nitrophenyl phosphate (8.9 mg/mL), HEDTA (0.7 mg/mL), zinc sulfate (0.29 mg/mL), and magnesium acetate (0.43 mg/mL) dissolved in 750 mM 2-amino-2-methyl-1-propanol solution (this is an aqueous solution at pH 10.2). After that, 20.0 μL of internal standard was added to each well. The resulting mixture in each well was mixed thoroughly by pipetting up and down 20-30× so as to precipitate proteins followed by centrifuging the mixture for about 10 minute at an RCF of about 5000 to provide a pellet at the bottom of each well.

Following protein precipitation, 50.0 μL from wells 1-12 of the first 96 well plate and 50.0 μL from wells 1-12 of the second 96 well plate were transferred to wells 1 to 12, respectively, of a third 96 well extraction plate containing 500 μL of acetonitrile in each well. After this, each well in the third plate was thoroughly mixed again by pipetting up and down 20-30 times followed by centrifuging for about 10 minutes at an RCF of about 5000. Finally, 300 μL of the supernatant from each of wells 1 to 12 of the third 96 well extraction plate was transferred to wells 1 to 12, respectively, of a fourth 96 well autosampler plate (commercially available from VWR International of Radnor, PA). The fourth plate was sealed and the contents of each of wells 1 to 12 analyzed using LC-MS/MS. Two injections were made of each well, for the first injection the mass spectrometer was operated in the negative ion mode and for the second injection the mass spectrometer was operated in the positive ion mode. The LC-MS/MS conditions are provided below.

### HPLC Conditions:

The assay was performed using a Shimadzu HPLC system (commercially available from Shimadzu Scientific Instruments of Columbia, MD) equipped with an Atlantis HILIC Column, 100 Å, 3 μm, 2.1 mm×100 mm (commercially available from Waters Corporation of Milford, MA) using the following gradient:
Mobile Phase A: 20 mM Ammonium formate in water, pH 3.5.
Mobile Phase B: 100% Acetonitrile.

For analysis when the mass spectrometer is configured to operate in the negative ion mode the following gradient was used:

Initial condition—95% B, 5% A; 0.1 min-95% B, 5% A; 1.3 min-30% B, 70% A; 1.4 min-95% B, 5% A; 2.5 min-95% B, 5% A; and stop the run, wherein the changes in solvent between timepoints was carried out using a linear gradient, and the column was eluted at a flow rate of 0.8 mL/min. i.e., HPLC conditions A.

For analysis when the mass spectrometer is configured to operate in the positive ion mode the following gradient was used:

Initial condition—80% B, 20% A; 0.1 min-80% B, 20% A; 0.7 min-80% B, 20% A; 0.71 min-30% B, 70% A; 1.2 min-30% B, 70% A; 1.21 min-80% B, 20% A; 2.5 min-80% B, 20% A; and stop the run, wherein the changes in solvent between timepoints was carried out using a linear gradient, and the column was eluted at a flow rate of 1.0 mL/min. i.e., HPLC conditions B.

The injection volume was 5.0 μL.

### Mass Spectrometer Configuration:

Analyses were performed using an ABSciex QTrap 5500 mass spectrometer (commercially available from SCIEX of Framingham, MA). When operated in the negative ion mode, the following configuration was used:

| a) | Scan Type: | MRM (MRM) | |
|---|---|---|---|
| b) | Scheduled MRM: | Yes | |
| c) | Polarity: | Negative | |
| d) | Scan Mode: | N/A | |
| e) | Ion Source: | Turbo Spray | |
| f) | Resolution Q1: | Unit | |
| g) | Resolution Q3: | Unit | |
| h) | Intensity Thres.: | 0.00 | cps |
| i) | Settling Time: | 0.0000 | msec |
| j) | MR Pause: | 5.0070 | msec |
| k) | MCA: | No | |
| l) | Step Size: | 0.00 | Da |

| | | |
|---|---|---|
| m) | Collision Gas (CAD): | MEDIUM |
| n) | Curtain Gas (CUR): | 25 |
| o) | Ion Source Gas1 (GS1): | 90 |
| p) | Ion Source Gas2 (GS2): | 60 |
| q) | lonSpray Voltage (ISV): | −4500 |
| r) | Temperature: | 600 |

and the spectrometer was configured to detect the ions in Table 1:

TABLE 1

| Parent | Daughter | | | | | | |
|---|---|---|---|---|---|---|---|
| ion | ion | Dwell | | | | | |
| (Q1) | (Q3) | Time | Compound being | | | | |
| m/z | m/z | (msec) | identified | DP ^{a} | EP ^{a} | CE ^{a} | CXP ^{a} |
| 137.92 | 46.1 | 50 | 4-Nitrophenol | −200 | −10 | −60 | −1 |
| | | | (i.e., the ALP product) | | | | |
| 143.92 | 46.1 | 50 | p-Nitrophenol labeled with | −200 | −10 | −60 | −1 |
| | | | ¹³C at each of the carbon | | | | |
| | | | atoms | | | | |
| | | | (i.e., the internal standard | | | | |
| | | | for ALP analysis) | | | | |
| 90.00 | 45.00 | 50 | Pyruvate labeled with ¹³C | −20 | −10 | −10 | −5 |
| | | | at the 1-, 2-, and 3-positions | | | | |
| | | | (i.e., the AST product) | | | | |
| 88.00 | 43.10 | 50 | Pyruvate labeled with ¹³C | −20 | −10 | −10 | −5 |
| | | | at the 1-position | | | | |
| | | | (i.e., the ALT product) | | | | |
| 89.00 | 44.00 | 50 | Pyruvate labeled with ¹³C | −20 | −10 | −10 | −5 |
| | | | at the 1- and 2-positions | | | | |
| | | | (i.e., the internal standard | | | | |
| | | | for ALT and AST | | | | |
| | | | analysis) | | | | |
| 178.92 | 118.80 | 50 | Glucose | −100 | −10 | −10 | −7 |
| 184.95 | 123.00 | 50 | Glucose labeled with ¹³C at | −100 | −10 | −10 | −7 |
| | | | each of the carbon atoms | | | | |
| | | | (i.e., the internal standard | | | | |
| | | | for glucose) | | | | |

^{a} DP means declustering potential, EP means entrance potential, CE means collision energy, and CXP means collision cell exit potential.

When operated in the positive ion mode, the following configuration was used:

| a) | Scan Type: | MRM (MRM) | |
|---|---|---|---|
| b) | Scheduled MRM: | No | |
| c) | Polarity: | Positive | |
| d) | Scan Mode: | N/A | |
| e) | Ion Source: | Turbo Spray | |
| f) | Resolution Q1: | Unit | |
| g) | Resolution Q3: | Unit | |
| h) | Intensity Thres.: | 0.00 | cps |
| i) | Settling Time: | 0.0000 | msec |
| j) | MR Pause: | 5.0070 | msec |
| k) | MCA: | No | |
| l) | Step Size: | 0.00 | Da |

| | | |
|---|---|---|
| m) | Collision Gas (CAD): | MEDIUM |
| n) | Curtain Gas (CUR): | 50 |
| o) | Ion Source Gas1 (GS1): | 60 |
| p) | Ion Source Gas2 (GS2): | 90 |
| q) | Ion Spray Voltage (ISV): | 1500 |
| r) | Temperature: | 700 |

and the spectrometer was configured to detect the ions in Table 2:

TABLE 2

| Parent | Daughter | | | | | | |
|---|---|---|---|---|---|---|---|
| ion | ion | Dwell | | | | | |
| (Q1) | (Q3) | Time | Compound being | | | | |
| m/z | m/z | (msec) | identified | DP ^{a} | EP ^{a} | CE ^{a} | CXP ^{a} |
| 113.97 | 44.20 | 50 | Creatinine | 150 | 10 | 20 | 5 |
| 113.97 | 86.10 | 50 | Creatinine | 100 | 10 | 20 | 10 |
| 117.00 | 47.10 | 50 | creatinine with a deuterated | 150 | 10 | 20 | 5 |
| | | | methyl group | | | | |
| | | | (i.e., the internal standard | | | | |
| | | | for creatinine) | | | | |
| 117.00 | 89.00 | 50 | creatinine with a deuterated | 100 | 10 | 20 | 10 |
| | | | methyl group | | | | |
| | | | (i.e., the internal standard | | | | |
| | | | for creatine) | | | | |
| 63.91 | 46.10 | 50 | Urea labeled with ¹³C and | 150 | 10 | 20 | 8 |
| | | | ¹⁵N | | | | |
| | | | (i.e., the internal standard | | | | |
| | | | for urea) | | | | |
| 60.91 | 44.10 | 50 | Urea | 150 | 10 | 20 | 8 |
| 203.07 | 46.10 | 50 | ADMA | 40 | 10 | 50 | 5 |
| 203.17 | 172.00 | 50 | SDMA | 20 | 10 | 20 | 5 |
| 210.22 | 46.20 | 2.01 | deuterated_ADMA | 40 | 10 | 50 | 5 |
| | | | (i.e., the internal standard | | | | |
| | | | for ADMA) | | | | |
| 209.20 | 175.20 | 1.99 | deuterated_SDMA | 20 | 10 | 20 | 5 |
| | | | (i.e., the internal standard | | | | |
| | | | for SDMA) | | | | |

^{a} DP means declustering potential, EP means entrance potential, CE means collision energy, and CXP means collision cell exit potential.

A standard curve is then created for each of the ALT product, the AST product, the ALP product, glucose, urea, creatinine, ADMA, and SDMA (i.e., the analytes) by plotting the intensity of the mass spectrometer peak for the daughter ion corresponding to the analyte relative to the intensity of the mass spectrometer peak for the daughter ion corresponding to the internal standard for that analyte vs. the concentration of the analyte. For example the standard curve for the ALT product is created by plotting the ratio of intensity of the peak at an m/z ratio of 43.10 relative to the intensity of the peak at an m/z ratio of 44.00 vs the concentration of ALT product.

### Determining Enzyme Activity in Serum Samples:

To measure AST and ALT activity of a serum sample 1, the following are added to a designated well of a first 96 well extraction plate.
1. 10.0 μL of oxaloacetic acid decarboxylase enzyme dissolved in 100 mM Tris-10 mM ammonium bicarbonate in water pH about 7.4. The solution of oxaloacetic acid decarboxylase is prepared to have a final activity level of 1000 U/L based on the certificate of analysis provided by the vendor. This is accomplished by adjusting the weight of the enzyme
2. 15.0 μL of alanine labelled with ¹³C in the 1-position dissolved in 100 mM Tris-10 mM ammonium bicarbonate in water at a pH of about 7.4. The concentration of alanine labelled with ¹³C in the 1-position is 55 mg/mL (prepared by dissolving 100 mg of alanine labelled with ¹³C in the 1-position in 1.8 mL of 100 mM Tris-10 mM Ammonium Bicarbonate in water at pH 7).
3. 65.0 μL of a solution containing pyridoxal 5′-phosphate monohydrate (0.1 mg/mL), α-ketoglutaric acid (8.8 mg/mL), and L-aspartic acid-¹³C4, 15N (3.1 mg/mL) in 100 mM Tris-10 mM ammonium bicarbonate in water pH about 7.4.

The first extraction plate was then covered and placed on an incubator for 20-30 minutes at 40° C. with shaking at 750 RPM to preheat the reaction mixture. After preheating, the plate was removed from the incubator and 10.0 μL of serum sample 1 was added to the designated well. After the sample was added, the plate was capped and immediately placed back on the incubator and incubated for 10 minutes at 40° C. with shaking at 750 RPM. Immediately after the 10 minute incubation period, the plate was removed from the incubator and 300 μL of methanol was added to the designated well of the first 96 well plate. Next, 20.0 μL of the internal standard was added to the designated well. The contents of the designated well of the first 96 well extraction plate were then mixed by pipetting up and down 20-30× to facilitate protein precipitation, followed by centrifuging for about 10 minutes at an RCF of about 5000 to provide a pellet at the bottom of the designated well. Several samples can be treated simultaneously, each sample having a unique designated well.

To measure ALP activity of serum sample 1, 90.0 μL of para-nitrophenyl phosphate solution (8.9 mg/mL), HEDTA (0.7 mg/mL), zinc sulfate (0.29 mg/mL), and magnesium acetate (0.43 mg/mL) in 750 mM 2-amino-2-methyl-1-propanol solution (this is an aqueous solution at pH 10.2) was added to a designated well of a second 96 well plate. The plate was then placed on an incubator to preheat the reaction mixture for 20-30 minutes at 40° C. with shaking at 750 RPM. After the plate was preheated, 10.0 μL of serum sample 1 was added to the designated well of the second 96 well plate. Upon sample addition, the plate was capped and immediately put back on the incubator and incubated for 10 minutes at 40° C. with shaking at 750 RPM. Immediately after the 10 minute incubation period, 300 μL of methanol was added to the designated well of the plate. After this, 20.0 L of internal standard was added to the designated well. Next, the contents of the designated well of the second 96 well plate were mixed by pipetting up and down 20-30× to facilitate protein precipitation, followed by centrifuging for about 10 minutes at an RCF of about 5000 to provide a pellet at the bottom of the plate. Several samples can be treated simultaneously, each sample having a unique designated well.

50.0 μL from the designated well of the first 96 well plate and 50.0 μL from the designated well of the second 96 well plate well were transferred to a designated well of a third 96 well extraction plate containing 500 μL of acetonitrile. The contents of the designated well of the third extraction plate were mixed by pipetting up and down 20-30x. The extraction plate was then centrifuged for about 12 minutes at an RCF of about 5000. 300 μL of the supernatant from the designated well of the third extraction plate was then transferred to a designated well of a fourth 96 well autosampler plate. The fourth plate was sealed and the contents of the designated well analyzed by LC-MS/MS as described above. Several samples can be treated simultaneously, each sample having a unique designated well.

The mass spectrometer, when operated in the negative ion mode, was configured to detect the ions in Table 3:

TABLE 3

| Parent | Daughter | | | | | | |
|---|---|---|---|---|---|---|---|
| ion | ion | Dwell | | | | | |
| (Q1) | (Q3) | Time | Compound being | | | | |
| m/z | m/z | (msec) | identified | DP ^{h} | EP ^{h} | CE ^{h} | CXP ^{h} |
| 137.92 | 46.1 | 50 | 4-Nitrophenol ^{a} | −200 | −10 | −60 | −1 |
| 143.92 | 46.1 | 50 | p-Nitrophenol labeled with | −200 | −10 | −60 | −1 |
| | | | ¹³C at each of the carbon | | | | |
| | | | atoms ^{b} | | | | |
| 90.00 | 45.00 | 50 | Pyruvate labeled with ¹³C | −20 | −10 | −10 | −5 |
| | | | at the 1-, 2-, and 3- | | | | |
| | | | positions ^{c} | | | | |
| 88.00 | 43.10 | 50 | Pyruvate labeled with ¹³C | −20 | −10 | −10 | −5 |
| | | | at the 1-position ^{d} | | | | |
| 89.00 | 44.00 | 50 | Pyruvate labeled with ¹³C | −20 | −10 | −10 | −5 |
| | | | at the 1- and 2-positions ^{e} | | | | |
| 178.92 | 118.80 | 50 | Glucose ^{f} | −100 | −10 | −10 | −7 |
| 184.95 | 123.00 | 50 | Glucose labeled with ¹³C at | −100 | −10 | −10 | −7 |
| | | | each of the carbon atoms ^{g} | | | | |

^{a} p-nitrophenol formed from the action of ALP on p-nitrophenyl phosphatase (i.e., the ALP product).
^{b} internal standard for ALP analysis.
^{c} pyruvate formed from the action of AST on aspartate labeled with ¹³C in the 1-, 2-, 3-, and 4-position and with ¹⁵N at the nitrogen (i.e., the AST product).
^{d} pyruvate formed from the action of ALT on alanine labelled with ¹³C at the 1-position (i.e., the ALP product).
^{e} internal standard for ALT and AST analysis.
^{f} glucose in the sample.
^{g} internal standard for glucose.
^{h} DP means declustering potential, EP means entrance potential, CE means collision energy, and CXP means collision cell exit potential.

The mass spectrometer, when operated in the positive ion mode, was configured to detect the ions in Table 4:

TABLE 4

| Parent | Daughter | | | | | | |
|---|---|---|---|---|---|---|---|
| ion | ion | Dwell | | | | | |
| (Q1) | (Q3) | Time | Compound being | | | | |
| m/z | m/z | (msec) | identified | DP ^{k} | EP ^{k} | CE ^{k} | CXP ^{k} |
| 113.97 | 44.20 | 50 | Creatinine ^{a} | 150 | 10 | 20 | 5 |
| 113.97 | 86.10 | 50 | Creatinine ^{b} | 100 | 10 | 20 | 10 |
| 117.00 | 47.10 | 50 | creatinine with a deuterated | 150 | 10 | 20 | 5 |
| | | | methyl group ^{c} | | | | |
| 117.00 | 89.00 | 50 | creatinine with a deuterated | 100 | 10 | 20 | 10 |
| | | | methyl group ^{d} | | | | |
| 63.91 | 46.10 | 50 | Urea labeled with ¹³C and | 150 | 10 | 20 | 8 |
| | | | ¹⁵N ^{e} | | | | |
| 60.91 | 44.10 | 50 | Urea ^{f} | 150 | 10 | 20 | 8 |
| 203.07 | 46.10 | 50 | ADMA ^{g} | 40 | 10 | 50 | 5 |
| 203.17 | 172.00 | 50 | SDMA ^{h} | 20 | 10 | 20 | 5 |
| 210.22 | 46.20 | 2.01 | deuterated_ADMA | 40 | 10 | 50 | 5 |
| 209.20 | 175.20 | 1.99 | deuterated_SDMA | 20 | 10 | 20 | 5 |

^{a} creatinine in the sample.
^{b} creatinine in the sample.
^{c} internal standard for creatinine
^{d} internal standard for creatinine
^{e} internal standard for urea.
^{f} urea in the sample.
^{g} ADMA in the sample.
^{h} SDMA in the sample.
ⁱ internal standard for ADMA.
^{j} internal standard for SDMA.
^{k} DP means declustering potential, EP means entrance potential, CE means collision energy, and CXP means collision cell exit potential.

In the above analysis:
4-nitrophenol and the internal standard for ALP analysis (i.e., p-nitrophenol labeled with ¹³C at each of the carbon atoms) had a retention time of about 0.41 min;
pyruvate labeled with ¹³C at the 1-, 2-, and 3-positions (i.e., pyruvate formed from the action of AST on aspartate labeled with ¹³C in the 1-, 2-, 3-, and 4-positions and with ¹⁵N at the nitrogen), pyruvate labeled with ¹³C at the 1-position (i.e., pyruvate formed from the action of ALT on alanine labelled with ¹³C at the 1-position), and pyruvate labeled with ¹³C at the 1- and 2-positions (i.e., the internal standard for ALT and AST analysis) had a retention time of about 1.07 min;
glucose and glucose labeled with ¹³C at each of the carbon atoms (i.e., the internal standard for glucose analysis) had a retention time of about 1.08 min;
urea and urea labeled with ¹³C and ¹⁵N (i.e., the internal standard for urea analysis) had a retention time of about 0.46 min;
creatinine and creatinine with a deuterated methyl group (i.e., the internal standard for creatinine analysis) had a retention time of about 0.68 min;
SDMA and deuterated SDMA (i.e., the internal standard for SDMA analysis) had a retention time of about 1.35 min; and
ADMA and deuterated ADMA (i.e., the internal standard for ADMA analysis) had a retention time of about 1.36 min.

The data was processed using Sciex OS software (commercially available from SCIEX of Framingham, MA).

### Calculation of Small Molecule Levels:

To determine the level of each small molecule (i.e., glucose, urea, creatinine, SDMA, and ADMA) the ratio of the intensity of the mass spectrum peak of the daughter ion for the small molecule relative to the intensity of the mass spectrum peak of the daughter ion of the internal standard for the small molecule was determined. For example, for urea the ratio of the intensity of the mass spectrum peak having an m/z of 44.10 was compared to the intensity of the mass spectrum peak having an m/z of 46.10. The concentration of urea that corresponded to this ratio was then determined from the calibration curve generated for urea, as described above.

### Calculation of Enzyme Activity:

The activity of each enzyme in the sample is calculated using the concentration of the product of the enzymatic reaction calculated from the calibration curves (in μg/mL) (determined in the same way that the concentration of each small molecule is determined), the molecular weight of the product, and an incubation time of 10 minutes. The enzyme activity is reported in “activity units per liter” (U/L, where U=μmol product/min). The equations provided below were used to calculate the enzyme activity for each enzyme in the sample. LC-MS/MS provides the concentration of the products formed by the enzymatic reaction in μg/mL. The Sciex OS software was programmed to automatically calculate the activity of each enzyme using the equations provided below and the concentration of the enzyme product determined from the calibration curve.

Equation for calculating AST enzyme activity:

$$\frac{( \frac{( \frac{μ g}{mL} \times 1000 )}{91 g / mol} )}{10 minutes} = U / L$$

Equation for calculating ALT enzyme activity:

$$\frac{( \frac{( \frac{μ g}{mL} \times 1000 )}{89 g / mol} )}{10 minutes} = U / L$$

Equation for calculating ALP enzyme activity:

$$\frac{( \frac{( \frac{μ g}{mL} \times 1000 )}{139.11 g / mol} )}{10 minutes} = U / L$$

### Results:

Three serum samples from three individual mice with diet induced non-Alcoholic SteatoHepatitis samples (NASH-1,-2, and-3) were analyzed using the above-described procedure. NASH-1,-2, and -3 were obtained from Taconic Biosciences of Germantown, NY.

The results of the analysis are provided in Table 5:

TABLE 5

| | Aspartate | Alanine | Alkaline | | | | | |
|---|---|---|---|---|---|---|---|---|
| | transaminase | transaminase | phosphatase | | | | | |
| Sample | activity | activity | activity | Glucose | Creatinine | Urea | SDMA | ADMA |
| ID | (U/L) | (U/L) | (U/L) | (mg/dL) | (mg/dL) | (mg/dL) | (ug/dL) | (ug/dL) |
| NASH-1 | 318 | 1128 | 323 | 367 | 0.08 | 95 | 2.9 | 21.7 |
| NASH-2 | 293 | 1056 | 296 | 337 | 0.06 | 87 | 3.0 | 19.8 |
| NASH-3 | 434 | 1515 | 277 | 323 | 0.07 | 88 | 2.9 | 16.8 |

The results show that each of ALT activity, AST activity, ALP activity, glucose, urea, creatinine, ADMA, and SDMA can conveniently and rapidly be determined in a single experiment that requires only two injections of the sample onto the HPLC column. Each sample can be analyzed in about 6 minutes (i.e., 2 injections with a less than 3 minute run time).

The entire disclosure of all references that have been cited are incorporated herein by reference.

What is claimed is:

1. A method for assaying for (i) one or more of alkaline phosphatase activity, alanine transaminase activity, aspartate transaminase activity and glucose levels and (ii) one or more of SDMA levels, urea levels, creatinine levels, and ADMA levels in a sample comprising:
(A) providing a sample suspected of containing one or more of alkaline phosphatase, alanine transaminase, aspartate transaminase and glucose and one or more of SDMA, urea, creatinine, and ADMA;
(B) dividing the sample into at least a first portion and a second portion;
(C) contacting
(i) the first portion with at least one of (a) a substrate for alanine transaminase, wherein the substrate for alanine transaminase comprises a first isotopic label and (b) a substrate for aspartate transaminase, wherein the substrate for aspartate transaminase comprises a second isotopic label; and
(ii) the second portion with a substrate for alkaline phosphatase;
(D) adding to the first portion or the second portion at least one of isotopically labelled SDMA comprising a seventh isotopic label; isotopically labelled glucose comprising a third isotopic label; isotopically labelled urea comprising a fourth isotopic label, isotopically labelled creatinine comprising a fifth isotopic label, or isotopically labelled ADMA comprising a sixth isotopic label;
(E) combining the first portion and the second portion to provide an assay mixture;
(F) passing a first quantity of the assay mixture through an HPLC column to provide a first eluant containing the components of the assay mixture, and
(a) analyzing at least a portion of the first eluant with a mass spectrometer, wherein the mass spectrometer is operated in the positive ion mode, under conditions to generate and detect at least one of:
(aa) an ion formed from the SDMA and an ion formed from the isotopically labelled SDMA, wherein the ion formed from the isotopically labelled SDMA comprises the seventh isotopic label;
(ab) an ion formed from the urea and an ion formed from the isotopically labelled urea, wherein the ion formed from the isotopically labelled urea comprises the fourth isotopic label;
(ac) an ion formed from the creatinine and an ion formed from the isotopically labelled creatinine, wherein the ion formed from the isotopically labelled creatinine comprises the fifth isotopic label; or
(ad) an ion formed from the ADMA and an ion formed from the isotopically labelled ADMA, wherein the ion formed from the isotopically labelled ADMA comprises the sixth isotopic label;
and
(G) passing a second quantity of the assay mixture through an HPLC column to provide a second eluant containing components of the assay mixture, and
(a) analyzing at least a portion of the second eluant with a mass spectrometer, wherein the mass spectrometer is operated in the negative ion mode, under conditions to generate and detect at least one of:
(aa) an ion formed from a metabolite resulting from the action of the alanine transaminase on the substrate for alanine transaminase, wherein the metabolite resulting from the action of the alanine transaminase on the substrate for alanine transaminase comprises the first isotopic label;
(ab) an ion formed from a metabolite resulting from the action of the aspartate transaminase on the substrate for aspartate transaminase, wherein the metabolite resulting from the action of the aspartate transaminase on the substrate for aspartate transaminase comprises the second isotopic label;
(ac) an ion formed from a metabolite resulting from the action of the alkaline phosphatase on the substrate for alkaline phosphatase; or
(ad) an ion formed from the glucose, and an ion formed from the isotopically labelled glucose, wherein the ion formed from the isotopically labelled glucose comprises the third isotopic label.

2. The method of claim 1, wherein the volume of the sample is less than about 50 μL.

3. The method of claim 1 wherein the sample is selected from the group consisting of blood, serum, plasma, urine, tissue homogenates, feces, sweat, saliva, spinal fluid, and synovial fluid.

4. The method of claim 3, wherein the sample is serum.

5. The method of claim 1, wherein:
the substrate for alanine transaminase is: 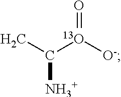
the substrate for aspartate transaminase is: 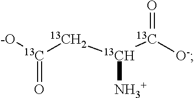
the substrate for alkaline phosphatase is p-nitrophenyl phosphate;
the isotopically labelled glucose is: 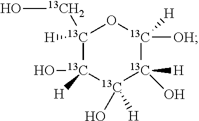 [SMILES: O[13CH2][13C@H]1O[13C@@H](O)[13C@H](O)[13C@@H](O)[13C@@H]1O]
the isotopically labelled urea is: 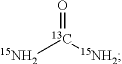
the isotopically labelled creatinine is: 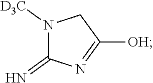 [SMILES: [2H]C([2H])([2H])N1CC(O)=NC1=N]
the isotopically labelled ADMA is: 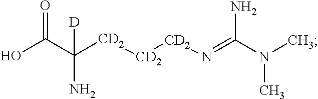
and
the isotopically labeled SDMA is:

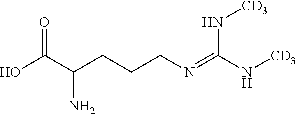 [SMILES: [2H]C([2H])([2H])NC(=NCCCC(N)C(=O)O)NC([2H])([2H])[2H]]

6. The method of claim 5, wherein the ion formed from a metabolite resulting from the action of the alanine transaminase on the substrate for alanine transaminase is detected by:
(i) ionizing the portion of the second eluant with the mass spectrometer to provide a plurality of parent ions,
(ii) separating the parent ions having an m/z ratio of 88 from the plurality of parent ions;
(iii) fragmenting the parent ions having an m/z ratio of 88 to provide a plurality of daughter ions;
(iv) separating daughter ions that have an m/z ratio of 43 from the plurality of daughter ions; and
(v) detecting the intensity of the daughter ions that have an m/z ratio of 43.

7. The method of claim 5, wherein the ion formed from a metabolite resulting from the action of the aspartate transaminase on the substrate for aspartate transaminase is detected by:
(i) ionizing the portion of the second eluant with the mass spectrometer to provide a plurality of parent ions,
(ii) separating the parent ions having an m/z ratio of 90 from the plurality of parent ions;
(iii) fragmenting the parent ions having an m/z ratio of 90 to provide a plurality of daughter ions;
(iv) separating daughter ions that have an m/z ratio of 45 from the plurality of daughter ions; and
(v) detecting the intensity of the daughter ions that have an m/z ratio of 45.

8. The method of claim 5, wherein the ion formed from a metabolite resulting from the action of the alkaline phosphatase on the substrate for alkaline phosphatase is detected by:
(i) ionizing the portion of the second eluant with the mass spectrometer to provide a plurality of parent ions,
(ii) separating the parent ions having an m/z ratio of 138 from the plurality of parent ions;
(iii) fragmenting the parent ions having an m/z ratio of 138 to provide a plurality of daughter ions;
(iv) separating daughter ions that have an m/z ratio of 46 from the plurality of daughter ions; and
(v) detecting the intensity of the daughter ions that have an m/z ratio of 46.

9. The method of claim 5, wherein contacting the first portion with a substrate for alanine transaminase further comprises contacting the first portion with α-ketoglutarate and pyridoxal phosphate.

10. The method of claim 5, wherein contacting the first portion with a substrate for aspartate transaminase further comprises contacting the first portion with α-ketoglutarate, pyridoxal phosphate, and oxaloacetate decarboxylase.

11. The method of claim 5, wherein contacting the second portion with a substrate for alkaline phosphatase comprises further contacting the second portion with zinc sulfate, magnesium acetate, and HEDTA.

12. The method of claim 1, comprising simultaneously assaying for (i) alkaline phosphatase activity, (ii) at least one of alanine transaminase activity and aspartate transaminase activity, (iii) at least one of urea levels, creatinine levels, ADMA levels, and SDMA levels, and (iv) optionally glucose levels.

13. The method of claim 1, comprising simultaneously assaying for each of alanine transaminase activity, aspartate transaminase activity, and alkaline phosphatase activity.

14. The method of claim 13, further comprising simultaneously assaying for each of glucose levels, urea levels, creatinine levels, ADMA levels, and SDMA levels.

15. The method of claim 5, wherein the ion formed from glucose and the ion formed from the isotopically labelled glucose are detected by:
(i) ionizing the portion of the second eluant with the mass spectrometer to provide a plurality of parent ions,
(ii) separating the parent ions having an m/z ratio of 179 and an m/z ratio of 185 from the plurality of parent ions,
(iii) fragmenting the parent ions having an m/z ratio of 179 and an m/z ratio of 185 to provide a plurality of daughter ions;
(v) separating daughter ions that have an m/z ratio of 119 and an m/z ratio of 123 from the plurality of daughter ions; and
(vi) detecting the intensity of the daughter ions that have an m/z ratio of 119 and an m/z ratio of 123.

16. The method of claim 5, wherein the ion formed from urea and the ion formed from the isotopically labelled urea are detected by:
(i) ionizing the portion of the first eluant with the mass spectrometer to provide a plurality of parent ions,
(ii) separating the parent ions having an m/z ratio of 61 and an m/z ratio of 64 from the plurality of parent ions;
(iii) fragmenting the parent ions having an m/z ratio of 61 and an m/z ratio of 64 to provide a plurality of daughter ions;
(iv) separating daughter ions that have an m/z ratio of 44 and an m/z ratio of 46 from the plurality of daughter ions; and
(v) detecting the intensity of the daughter ions that have an m/z ratio of 44 and an m/z ratio of 46.

17. The method of claim 5, wherein the ion formed from urea and the ion formed from the isotopically labelled urea are detected by:
(i) ionizing the portion of the first eluant with the mass spectrometer to provide a plurality of parent ions,
(ii) separating the parent ions having an m/z ratio of 114 and an m/z ratio of 117 from the plurality of parent ions,
(iii) fragmenting the parent ions having an m/z ratio of 114 and an m/z ratio of 117 to provide a plurality of daughter ions;
(iv) separating daughter ions that have an m/z ratio of 44 and an m/z ratio of 47 from the plurality of daughter ions or separating daughter ions having an m/z ratio of 86 and 89 from the plurality of daughter ions; and
(v) detecting the intensity of the daughter ions that have an m/z ratio of 44 and an m/z ratio of 47 or detecting the intensity of the daughter ions that have an m/z ratio of 86 and an m/z ratio of 89.

18. The method of claim 5, wherein the ion formed from ADMA and the ion formed from the isotopically labelled ADMA are detected by:
(i) ionizing the portion of the first eluant with the mass spectrometer to provide a plurality of parent ions,
(ii) separating the parent ions having an m/z ratio of 203 and an m/z ratio of 210 from the plurality of parent ions;
(iii) fragmenting the parent ions having an m/z ratio of 203 and an m/z ratio of 210 to provide a plurality of daughter ions;
(iv) separating daughter ions that have an m/z ratio of 46 from the plurality of daughter ions; and
(v) detecting the intensity of the daughter ions that have an m/z ratio of 46.

19. The method of claim 5, wherein the ion formed from SDMA and the ion formed from the isotopically labelled SDMA are detected by:
(i) ionizing the portion of the first eluant with the mass spectrometer to provide a plurality of parent ions,
(ii) separating the parent ions having an m/z ratio of 203 and an m/z ratio of 209 from the plurality of parent ions,
(iii) fragmenting the ions having an m/z ratio of 203 and an m/z ratio of 209 to provide a plurality of daughter ions;
(iv) separating daughter ions that have an m/z ratio of 172 and an m/z ratio of 175 from the plurality of daughter ions; and
(v) detecting the intensity of the daughter ions that have an m/z ratio of 172 and an m/z ratio of 175.
